(19) United States
(12) Patent Application Publication
(10) Pub. No.: US 2026/0209366 A1
(43) Pub. Date: Jul. 23, 2026

(54) METHODS, KITS AND COMPOSITIONS FOR THE TREATMENT OF FOOD PROTEIN INDUCED ENTEROCOLITIS SYNDROME AND IRRITABLE BOWEL SYNDROME

(71) Applicants: Oral ALPAN, McLean, VA (US); Matthew PLASSMEYER, Sterling, VA (US)

(72) Inventors: Oral ALPAN, McLean, VA (US); Matthew PLASSMEYER, Sterling, VA (US)

(73) Assignee: Amerimmune, McLean, VA (US)

(21) Appl. No.: 19/313,695

(22) Filed: Aug. 28, 2025

Related U.S. Application Data
(60) Provisional application No. 63/688,340, filed on Aug. 29, 2024.
(60) Provisional application No. 63/742,120, filed on Jan. 6, 2025.
(60) Provisional application No. 63/828,614, filed on Jun. 23, 2025.
(60) Provisional application No. 63/839,282, filed on Jul. 7, 2025.

(51) Int. Cl. C07K 16/28; A61K 39/00; A61P 1/00; A61P 37/08; G01N 15/01

(52) U.S. Cl. CPC C07K 16/2866; A61P 1/00; A61P 37/08; G01N 15/01; A61K 2039/505; A61K 2039/545; C07K 2317/21; C07K 2317/76

(57) ABSTRACT

This invention describes methods, kits, and compositions for treating Food Protein-Induced Enterocolitis Syndrome (FPIES) and a food-triggered endotype of IBS by modulating the IL-4/IL-13→IL-4Rα axis and/or the OX40-OX40L costimulatory pathway, with patient selection and monitoring guided by blood biomarkers (elevated OX40L on dendritic cells and/or OX40 on lymphocytes, including food antigen-stimulated assays). In case examples, IL-4Rα blockade (e.g., dupilumab) produced rapid, durable clinical remission with successful food reintroduction, accompanied by reduced dendritic-cell OX40L and modulation of circulating CRTH2⁺ Tc2 cells, whereas OX40L-low, non-food-trigger IBS showed no response—supporting a biomarker-defined responder population. Collectively, this invention demonstrates a precision framework in which IL-4Rα and OX40/OX40L antagonists—alone or in combination—enable diet liberalization and induce tolerance in FPIES and food-triggered IBS.

### RELATED APPLICATIONS

This application and the inventions described herein are related to U.S. provisional patent application Ser. Nos. 63/688,340, filed on Aug. 29, 2024, 63/742,120 filed on Jan. 6, 2025, 63/828,614, filed on Jun. 23, 2025 and 63/839,282 filed on Jul. 7, 2025 respectively, and the inventions described therein, each of which applications are hereby incorporated by reference in its entirety for all purposes.

### BACKGROUND

### Overview and Nature of Food Protein-Induced Enterocolitis Syndrome

Food Protein-Induced Enterocolitis Syndrome (FPIES) is a non-IgE-mediated immune response to food proteins that primarily affects the gastrointestinal tract. It is distinct from classic IgE-mediated food allergies in that symptoms are delayed, typically appearing 1-4 hours after ingestion of the triggering food, and are dominated by gastrointestinal manifestations rather than cutaneous or respiratory signs. FPIES can occur in both children and adults, though it is most often diagnosed in infancy, frequently after the introduction of formula or solid foods. A single patient may react to one or multiple foods, and the specific food triggers can vary widely between individuals.

### Common Triggers

Cow's milk and soy are the most frequent culprits in infants, especially those who react to formula. In toddlers and older children, solid foods such as rice, oats, wheat, barley, poultry, fish, shellfish, and certain vegetables (e.g., sweet potato, peas) have been reported. Multiple food triggers are common, and in some cases, the range of reactive foods expands over time. Less commonly, adults may develop FPIES to seafood, dairy, peanuts and tree nuts or grains, often with a history of tolerance earlier in life. All ingested foods can be a culprit in pediatric or adult patients.

### Acute Presentation

The hallmark of acute FPIES is repetitive, projectile vomiting starting 1-4 hours after ingestion of the offending food. This is often followed by watery diarrhea that may develop several hours later, occasionally containing mucus or blood. Some patients can appear pale, lethargic, and may be hypotensive in severe reactions. Some develop hypothermia or go into shock, requiring emergency care. The absence of immediate skin symptoms (urticaria, angioedema) or respiratory compromise differentiates acute FPIES from anaphylaxis and food allergies, although the circulatory collapse in severe cases can be equally dangerous.

### Chronic/Atypical Presentation

In chronic FPIES, seen when the offending food is ingested regularly (e.g., daily formula feeding), the presentation is more insidious. Infants may develop intermittent vomiting, persistent diarrhea, poor feeding, irritability, and failure to thrive. Growth faltering can be significant if the trigger is not eliminated. Because symptoms are not tied to a single acute ingestion, diagnosis in chronic cases can be delayed, sometimes leading to misdiagnosis as gastroesophageal reflux, infection, or other gastrointestinal disorders. Adult can manifest as abdominal pain, loose stool, diarrhea, blood in stool, heartburn, abdominal distention, irritable bowel-like symptoms and vomiting.

### Laboratory and Diagnostic Findings

During acute reactions, laboratory tests may reveal marked neutrophilia within hours, with white blood cell counts sometimes doubling from baseline. Thrombocytosis is common in chronic FPIES, and metabolic derangements such as metabolic acidosis or methemoglobinemia can occur in severe cases. Serum IgE testing and skin prick testing are typically negative for the trigger food, distinguishing them from food allergies.

### Natural History and Prognosis

Many children outgrow FPIES to milk or soy by age 3-5 years, though reactions to grains, seafood, and multiple foods often persist longer. Adult-onset FPIES is increasingly recognized and may persist indefinitely without tolerance development. Spontaneous remission in adults is rare. The severity and unpredictability of reactions, combined with the potential for multiple triggers, makes long-term dietary management challenging.

### Differentiation from Other Disorders

The delayed onset and lack of cutaneous or respiratory features distinguish FPIES from IgE-mediated anaphylaxis and food allergies. However, the hemodynamic instability in severe cases underscores that FPIES can be equally life-threatening. Its episodic nature can lead to misdiagnosis as viral gastroenteritis or sepsis, particularly when the temporal relationship to food ingestion is not recognized.

### Differentiation of FPIES from Eosinophilic Gastrointestinal Diseases (EGIDs)

FPIES and EGIDs are both food-triggered GI inflammatory diseases but diverge sharply in behavior and management. FPIES causes delayed, acute reactions-repetitive vomiting 1-4 hours after a trigger with diarrhea later-often with systemic toxicity (pallor, lethargy, dehydration, even hypotension), negative IgE tests, and nonspecific biopsies without dense eosinophilia; patients are usually well between episodes in the acute form, many children outgrow milk/soy triggers, and treatment is strict avoidance plus supportive care (no approved drugs). By contrast, EGIDs produce chronic, insidious symptoms tied to ongoing exposure (pain, nausea, bloating, diarrhea; dysphagia in Eosinophilic Esophagitis [EoE]), rarely cause systemic collapse but can lead to weight loss/nutritional deficits, often coexist with atopy and positive IgE sensitization, and show eosinophil-predominant tissue infiltration (e.g., ≥15 eos/hpf in EoE) with remodeling/fibrosis. EGIDs are typically relapsing and persistent without sustained therapy and respond to elimination diets and corticosteroids; dupilumab is effective for EoE, and remission generally requires ongoing dietary or pharmacologic control. Key Clinical Distinction: The episodic, acute presentation of FPIES with complete resolution between reactions (in the acute form), and the absence of histologic eosinophilia, distinguishes it from the chronic, tissue eosinophil-driven pathology of EGIDs. While both share elements of Th2-mediated inflammation, the effector mechanisms and temporal patterns differ sufficiently to require different diagnostic workups and treatment paradigms.

### Pathophysiology

The pathophysiology of FPIES has been poorly understood. Although originally classified as a prototypical delayed-type (Type IV) hypersensitivity reaction, emerging evidence shows that FPIES is not driven by a single immunologic pathway but rather represents a mixed immune disease in which multiple innate and adaptive mechanisms operate concurrently. This complexity makes it inherently difficult to predict the optimal therapeutic pathway for intervention. Acute FPIES reactions demonstrate contributions from both the innate and adaptive immune systems. Innate activation is reflected in elevated neutrophil counts, increased serum IL-8, TNF-α, and other proinflammatory cytokines during episodes, implicating early recruitment of inflammatory cells to the gut mucosa. Adaptive responses are evidenced by antigen-specific T-cell activation, with skewing toward a type 2 helper T cell (Th2) profile in many patients, characterized by IL-5 and IL-13 production, despite the absence of IgE antibodies to the triggering food.

While Th2 cytokines (IL-4, IL-5, IL-13) are increasingly recognized as drivers of mucosal inflammation in FPIES, several studies have also documented Th1 cytokine involvement (e.g., IFN-γ), particularly in chronic forms of the disease or in recovery phases, as well as Th17-related mediators (IL-17A) in some patients. Regulatory T cell (Treg) dysfunction and altered IL-10 production have been reported, further complicating the immune signature. The relative dominance of these pathways appears to vary not only between patients but also within the same patient over time or depending on the triggering antigen.

Peripheral blood mononuclear cells from different FPIES patients show heterogeneous cytokine release patterns when stimulated with culprit foods ex vivo-some display strong Th2 cytokine induction, others show mixed Th1/Th2 signatures, and some primarily exhibit innate cytokine responses. This variability suggests that no single pathway is universally dominant in all FPIES patients.

Because FPIES pathophysiology encompasses both innate and adaptive immunity, with contributions from multiple T-cell subsets and antigen-presenting cell interactions, the disease does not map neatly onto one known therapeutic axis.

### How FPIES is Currently being Treated

There is currently no approved pharmacotherapy specifically for FPIES. The management primarily consists of avoidance of the specific food triggers or supportive care during an acute episode.

Acute Reaction (clinic/ED)
Treat as a potential emergency; ~15% can develop hypovolemic shock. Start aggressive isotonic IV fluids (10-20 mL/kg boluses, repeat as needed, then maintenance).
Ondansetron is the antiemetic of choice: 0.15 mg/kg IM/IV (max 16 mg/dose); may avert progression of minor criteria during OFC and hasten recovery.
Consider IV methylprednisolone 1 mg/kg (max 60-80 mg) for persistent emesis/inflammation (low-quality evidence). Monitor/correct acid-base, electrolytes; treat methemoglobinemia if present.

### Home Action Plan (Carefully Selected Patients)

Oral rehydration at first symptoms (clear fluids/breast-feeding). Oral ondansetron 0.15 mg/kg can be used at home when provided and trained; escalate/activate EMS for ≥3 emesis episodes, lethargy, or any red flags.

### Long-Term Management

Strict avoidance of confirmed triggers; provide dietitian support to maintain growth and dietary variety. Avoid blanket avoidance of entire food groups.
Breast-feeding: continue if infant is well; do not routinely place the lactating parent on an elimination diet unless the breast-fed infant is symptomatic. If symptoms occur with exclusive breast-feeding, consider maternal elimination under specialist guidance.
Formula choice (CM-triggered FPIES):
   Start with extensively hydrolyzed formula (eHF) if needed.
   Use amino acid formula (AAF) when there's severe acute/chronic FPIES, faltering growth, persistent symptoms on, multiple severe GI allergies, or other high-risk scenarios.
   Soy co-reactivity occurs in ~20-40% of US CM-FPIES; goat/sheep milks are not suitable alternatives.

### Re-Introduction/Oral Food Challenge (OFC)

Re-evaluate periodically (commonly 12-18 months after last reaction); timing varies by age and trigger. Perform supervised OFCs (consider IV access for severe histories).
Natural history (pediatrics): tolerance to CM/soy often earlier than to grains; pooled data suggest many CM-FPIES resolve by ~3 years (e.g., 60% by 1 y, 75% by 2 y, 85% by 3 y in one cohort), though US series report later medians and wide variability-hence the need for individualized re-testing.
Home reintroduction may be reasonable for carefully preselected mild cases with training and clear plans; early data show high success and only mild reactions at home. Use shared decision-making.

### Food-Triggered Irritable Bowel Syndrome (IBS) that Overlaps with FPIES

Approximately 40% IBS patients (Diarrhea common IBS [IBS-D], diarrhea/constipation alternating IBS [IBS-M], and post-infectious IBS) report reproducible symptoms after specific foods (e.g., wheat/gluten, soy, milk proteins) despite negative standard allergy testing. These cases can show mucosal/peripheral immune activation and are frequently managed with dietary avoidance-yet many remain symptomatic.

Adult FPIES (and late-teen cases) are under-recognized and often coded clinically as “IBS with food triggers,” given the delayed, non-IgE GI manifestations (nausea, emesis, diarrhea, crampy pain, pallor, lethargy) after a culprit food, with normal endoscopy between episodes.

### Core Diagnostic Rule of IBS (Rome IV):

Recurrent abdominal pain ≥1 day/week in the last 3 months, with symptom onset ≥6 months before diagnosis, associated with ≥2 of:
1. related to defecation,
2. change in stool frequency,
3. change in stool form (Bristol scale).

### Subtypes (by Bristol Stool Forms on Abnormal-Stool Days)

IBS-C: >25% types 1-2 and <25% types 6-7
IBS-D: >25% types 6-7 and <25% types 1-2
IBS-M: >25% types 1-2 and >25% types 6-7
IBS-U: criteria met, but stool pattern not abnormal enough for subtyping.

As used herein, ‘Rome-style IBS’ means irritable bowel syndrome meeting Rome IV criteria: recurrent abdominal pain on average at least once weekly during the prior 3 months, with symptom onset ≥6 months before diagnosis, associated with at least two of: relation to defecation, change in stool frequency, or change in stool form; subtyped as IBS-C, IBS-D, IBS-M, or IBS-U per Rome IV Bristol stool thresholds.

The Bristol Stool Form Scale (BSFS) is a 7-point chart that describes stool shape/consistency. It's used clinically (and in research/patents) to standardize bowel-habit reporting and to subtype IBS.
1. Type 1: separate hard lumps, like nuts—severe constipation
2. Type 2: sausage-shaped but lumpy—mild constipation
3. Type 3: sausage with cracks on the surface—normal-ish
4. Type 4: smooth, soft sausage/snake—ideal/normal
5. Type 5: soft blobs with clear-cut edges—borderline loose
6. Type 6: fluffy pieces with ragged edges, mushy—diarrhea
7. Type 7: watery, no solid pieces—severe diarrhea

IBS is understood as a disorder of gut-brain interaction with contributions from visceral hypersensitivity, disordered motility, altered intestinal permeability, mucosal immune activation, microbial dysbiosis, bile-acid malabsorption in a subset, and psychosocial factors. Post-infectious IBS is a recognized entity following acute gastroenteritis. There is an overlap between FPIES and IBS, where the symptom of IBS is triggered with specific foods, suggesting an immune mechanism. Many patients report symptom exacerbations related to meals or specific foods. This is different than non-immune mechanisms where dietary carbohydrates that are poorly absorbed and highly fermentable increasing luminal water and gas, provoking pain, bloating, and altered stool form. The immune mediated food triggered gastrointestinal symptoms are reproducible, delayed after foods (e.g., wheat or milk) despite negative IgE-mediated allergy tests; these patterns are often managed clinically as “food-triggered IBS” and may overlap with other non-IgE food-related conditions such as FPIES.

Diagnosis of IBS is made positively using symptom criteria with limited testing to exclude alternative diagnoses when indicated by alarm features (e.g., weight loss, anemia, nocturnal symptoms, gastrointestinal bleeding, family history of IBD or colorectal cancer, onset at an older age). Common evaluations include celiac serology in IBS-D or IBS-M and inflammatory markers (e.g., fecal calprotectin) when inflammatory bowel disease is a concern. Endoscopy is reserved for age-appropriate cancer screening or when alarm features are present.

### How IBS is currently Treated

Current management is individualized and multimodal. Non-pharmacologic strategies include patient education, dietary modification, and gut-directed psychotherapies. Pharmacologic therapy is typically subtype-directed: antidiarrheals or rifaximin, eluxadoline, and (in selected women) alosetron for IBS-D; secretagogues (linaclotide, plecanatide, lubiprostone), tenapanor, or prokinetics for IBS-C; and low-dose tricyclic antidepressants for global symptom and pain modulation across subtypes. Despite these approaches, a substantial proportion of patients remain symptomatic or experience adverse effects, reflecting the heterogeneity of underlying mechanisms and the absence of widely adopted biomarkers to guide selection of therapy. Food-related symptoms overlap with Rome criteria. For example, a patient may consistently experience abdominal pain and altered stool after eating wheat or dairy; this still qualifies as IBS under Rome if the core criteria are met, but clinicians might label it “meal-related IBS” or “food-triggered IBS.” Approximately 60-70% of all IBS patients identify specific foods as symptom triggers.

Even though Rome criteria don't mention diet, management guidelines explicitly recommend dietary interventions (targeted elimination, reintroduction) as first-line treatment for many patients.

Research suggests food-triggered IBS may represent a biologically distinct endotype, where immune responses to food antigens (mast cell activation, OX40L signaling, mucosal immune priming) contribute to pain and diarrhea. This mechanistic angle is not yet part of Rome, but could eventually support refining classification (e.g., “immune-mediated food-sensitive IBS”).

### Clinical Response

In subjects with FPIES and/or food-triggered irritable bowel syndrome IBS or IBS clinical improvement is defined a priori by one or more of the following: for FPIES—≥50% reduction in delayed emesis/diarrhea or any gastrointestinal symptom episodes within 1-8 hours or the delayed responses in atypical FPIES after culprit food exposure, elimination of emergency/urgent-care visits, and/or increased tolerated dose on oral food challenge by ≥2-fold or complete tolerance; and for IBS—≥30% reduction from baseline in worst daily abdominal pain in ≥30% of treatment weeks and normalization of stool form (Bristol 3-5) in ≥30% of weeks, and/or ≥30-point decrease in IBS-Symptom Severity Score (IBS-SSS); global response—Patient Global Impression of Change of “much improved” or “very much improved” at ≥2 consecutive visits; functional/health impact—≥3-point improvement in PROMIS GI scales or—≥20-50% decrease from baseline in dendritic-cell OX40L expression.

### Compounds

### Dupilumab is an Interleukin-4 Receptor Alpha Antagonist Indicated for:

Atopic Dermatitis: for the treatment of adult and pediatric patients aged 6 months and older with moderate-to-severe AD whose disease is not adequately controlled with topical prescription therapies or when those therapies are not advisable. Dupilumab can be used with or without topical corticosteroids. For adults the dose is an initial 600 mg loading dose followed by 300 mg every 2 weeks. For pediatric patients, depending on the body weight, it between 200-300 mg every 4 weeks with a 400-600 mg initial loading dose. Specifically: for 6 months to 5 years of age; 200 mg Q2W (5-15 kg), 300 mg Q2W (15-30 kg). For 6-17 years of age; 600 mg loading followed by 300 mg Q4W (15-30 KG), 400 mg loading followed by 200 mg Q2W (30-60 kg), 600 mg loading followed by 300 mg Q2W (>60 kg).

Asthma as an add-on maintenance treatment of adult and pediatric patients aged 6 years and older with moderate-to-severe asthma characterized by an eosinophilic phenotype or with oral corticosteroid dependent asthma. Limitations of Use: Not for the relief of acute bronchospasm or status asthmaticus. For adult patients a loading dose between 400-600 mg, with subsequent dosing of 200-300 mg every 2 weeks. In pediatric patients 6-11 years of age 200 every 2 weeks (>30 kg) or 300 mg every 4 weeks (15-30 kg) depending on the body weight. Chronic Rhinosinusitis with Nasal Polyps: as an add-on maintenance treatment in adult and pediatric patients aged 12 years and older with inadequately controlled chronic rhinosinusitis with nasal polyps (CRSwNP). The dose in pediatric and adult patients are 300 mg every other week without a loading dose.

Eosinophilic Esophagitis: for the treatment of adult and pediatric patients aged 1 year and older, weighing at least 15 kg, with eosinophilic esophagitis (EoE). The dosing is 200 mg Q2W (15-30 kg), 300 mg QOW (30-40 kg) or 300 mg weekly (>30 kg).

Prurigo Nodularis: for the treatment of adult patients with prurigo nodularis (PN). The dose is 600 mg loading dose followed by 300 mg Q2W.

Chronic Obstructive Pulmonary Disease: as an add-on maintenance treatment of adult patients with inadequately controlled chronic obstructive pulmonary disease (COPD) and an eosinophilic phenotype. Limitations of Use: Not for the relief of acute bronchospasm. The dose is 300 mg Q2W.

Some of the medications involved in the current invention target either the IL-4Rα receptor or the OX40/OX40L, are in clinical development and not yet approved by the FDA:

For each agent, the table provides its generic name (and/or code name), target, sponsor/developer, route of administration, typical dosing regimen (as per clinical use or trials), and current development phase or approval status. All these agents are contemplated in various embodiments of the invention for treating FPIES or related conditions (Table 1).

TABLE 1
IL-4Rα and OX40/OX40L Pathway-Targeting Therapeutics Relevant to the Invention [81][82]

| | Sponsor/ | Route & | Development | |
|---|---|---|---|---|
| Drug (Target) | Developer | Dosing | Status | Notes/Indications |
| Rademikibart (IL- | Connect | Subcutaneous. | Phase 3 (atopic | “Next-gen” IL-4Rα |
| 4Rα mAb, a.k.a. | Biopharma | Trials in AD | dermatitis)[48]; | antibody with ~2× |
| CBP-201) | | used 150 mg or | Phase 2 | binding affinity vs |
| | | 300 mg Q2W, | (asthma) | dupilumab [85]. |
| | | and exploring | ongoing. Not | Showed ~63% |
| | | Q4W dosing due | yet FDA | EASI-75 in AD at 16 |
| | | to high | approved. | wks (higher than |
| | | affinity[36][50]. | | dupilumab)[48]. |
| | | | | Potential monthly |
| | | | | dosing. |
| Manfidokimab (IL- | Akeso Biopharma | Subcutaneous or | Phase 3 | Humanized IgG4 |
| 4Rα mAb, a.k.a. | (China) | IV. Phase 2 AD | (China, | targeting IL- |
| AK120) | | trial AK120-201 | moderate- | 4Rα[82]. Designed |
| | | used doses up to | severe | to block IL-4/13 |
| | | ~300 mg Q2W | AD)[54]; | signaling like |
| | | (exact regimen | Phase 2 (global | dupilumab. Asthma |
| | | TBD). | AD)[86]. Not | program |
| | | | yet approved. | discontinued for |
| | | | | strategic |
| | | | | reasons [54]. AD |
| | | | | trials ongoing with |
| | | | | favorable safety. |
| Rocatinlimab (Anti- | Kyowa Kirin/ | Subcutaneous. | Phase 3 (atopic | Fully human IgG1 |
| OX40 mAb, a.k.a. | Amgen | Phase 2b AD | dermatitis) | anti-OX40[37] that |
| KHK4083/AMG 451) | | regimens: | ongoing. Not | depletes OX40 + T |
| | | 150 mg Q4W, | yet approved. | cells. In AD Phase |
| | | 600 mg Q4W, | | 2b, all active dose |
| | | 300 mg Q2W, or | | arms showed >50% |
| | | 600 mg Q2W; | | EASI improvement |
| | | with or without | | vs ~15% |
| | | loading | | placebo[36][87]. |
| | | doses[36]. | | Durable responses |
| | | Likely Q4W in | | observed even after |
| | | current trials. | | stopping drug, |
| | | | | hinting at disease |
| | | | | modification. |
| Telazorlimab (Anti- | Ichnos Sciences | Subcutaneous. | Completed | Humanized IgG1 |
| OX40 mAb, a.k.a. | (spun from | Tested | Phase 2b | anti-OX40. Well- |
| ISB 830/GBR 830) | Glenmark) | regimens: 75 mg | (AD)[88]; no | tolerated in trials; |
| | | Q4W, 300 mg | Phase 3 | 300 mg Q2W |
| | | Q4W, 300 mg | ongoing as of | significantly |
| | | Q2W, 600 mg | 2024[89]. | improved AD (54% |
| | | Q2W (with | Licensed to | EASI ↓ vs 34% in |
| | | initial loading | Astria for | placebo)[57]. |
| | | for Q2W | further | Development in |
| | | arms)[77][78]. | development. | limbo (partnering/ |
| | | | | licensing) but a |
| | | | | candidate for food |
| | | | | allergy uses in |
| | | | | this invention. |
| Amlitelimab (Anti- | Kymab (Sanofi) | Subcutaneous. | Phase 2b | Fully human IgG1 |
| OX40L mAb, a.k.a. | | Phase 2a AD: | completed | anti-OX40 Ligand |
| KY1005/SAR445229) | | 100 mg Q4W or | (AD)[93] with | (blocks |
| | | 250 mg Q4W | positive | OX40L)[61]. Non- |
| | | (with loading | results; entered | depleting (targets |
| | | dose for | Phase 3 (AD) | APCs). In AD trials, |
| | | 250 mg)[39][90]. | in 2023[94]. | showed dose- |
| | | Phase 2b tested | Not yet | dependent efficacy |
| | | doses 62.5-250 | approved. | and reduced IL-22, |
| | | mg Q4W[91][92]. | | with good |
| | | | | safety[64][95]. |
| | | | | Could prevent Th2 |
| | | | | priming by APCs in |
| | | | | food allergy. |

This table highlights that there are multiple agents capable of modulating these pathways, giving clinicians a toolkit to choose from. The treatments described in this invention anticipates the use of any such agent (including biosimilars or derivatives that may be developed) in treating FPIES. For instance, if a patient does not tolerate one drug, an alternative targeting the same pathway can be used. Combination or sequential use (e.g., induce remission with one agent, maintain with another) is within scope.

### BRIEF DESCRIPTION

One embodiment is directed to a method of treating Food Protein-Induced Enterocolitis Syndrome (FPIES) in a human patient in need thereof, comprising administering to the patient a therapeutically effective amount of an interleukin-4 receptor alpha (IL-4Rα) antagonist. In the method: FPIES may exists with one or more of atopic dermatitis, type 2 asthma, eosinophilic esophagitis, chronic spontaneous urticaria, prurigo nodularis or chronic rhino sinusitis with polyps (CRSwNP). The IL-4Rα antagonist may be a monoclonal antibody that binds and blocks IL4 and IL13 signaling through the IL-4Rα receptor. The IL-4Rα antagonist may be dupilumab, administered as a subcutaneous injection (for example, about 200-600 mg every two or four weeks in an adult or weight-adjusted equivalent in a child). The IL-4Rα antagonist may be dupilumab, administered as a subcutaneous injection at a dose of between 200-300 mg. The IL-4Rα antagonist may be rademikibart (also known as CBP-201), administered subcutaneously at a dose of about 150-300 mg every one to four weeks. The IL-4Rα antagonist may be manfidokimab (AK120); (for example, 200-300 mg every two weeks).

Another embodiment is directed to a method of treating FPIES in a human patient in need thereof, comprising administering a therapeutically effective amount of an OX40 or OX40L pathway inhibitor. In the method: The OX40/OX40L pathway inhibitor may be a monoclonal antibody that binds OX40 to prevent its interaction with OX40L. The antibody against OX40 may be rocatinlimab, administered subcutaneously at a dose of about 150 mg to 600 mg every 2 or 4 weeks (with or without an initial loading dose). The antibody against OX40 may be telazorlimab, administered subcutaneously at a dose of about 300 mg every two weeks or 300 mg every four weeks (following a loading dose), or about 600 mg every two weeks. The OX40L pathway inhibitor may be a monoclonal antibody that binds OX40L (OX40 ligand) to prevent its interaction with OX40. The antibody against OX40L may be amlitelimab, administered via subcutaneous injection at a dose of about 100 mg to 250 mg every four weeks (optionally with an initial loading dose of 200-500 mg). The patient may be administered both (a) an IL-4Rα antagonist and (b) an OX40 or OX40L pathway inhibitor (either concurrently as combination therapy, or sequentially within an overlapping treatment regimen). The IL-4Rα antagonist may be selected from the group consisting of dupilumab, rademikibart and manfidokimab, the OX40 pathway inhibitor selected from the group consisting of rocatinlimab and telazorlimab, and the OX40L pathway inhibitor may be amlitelimab.

Another embodiment is directed to a method of inducing oral tolerance to a food allergen in a patient with FPIES comprising: (a) administering, to the patient a therapeutically effective amount of IL4Rα antagonist, and (b) administering the food allergen in increasing amounts. In the method: Step (b) may comprise controlled exposure via oral immunotherapy protocol or gradual dietary introduction, and step (a) may be administering the IL-4Rα antagonist given biweekly throughout the protocol, and wherein tolerance may be assessed by negative food challenge after at least 3-6 months of therapy. Step (a) may be administering OX40 or OX40L pathway inhibitor.

Another embodiment is directed to a method of determining whether an FPIES patient would benefit from IL4Rα antagonist or OX40L/OX40 pathway inhibitor treatment by testing the patient's blood sample using flow cytometry to determine if; (a) ≥2% of events within either the CD11c⁺ myeloid dendritic-cell gate or the CD303 (BDCA-2)⁺ plasmacytoid dendritic-cell gate exhibiting OX40L signal above a positivity threshold set by a fluorescent-minus-one (FMO) control in the same assay of an age- and sex-matched healthy reference distribution measured on the same instrument, antibody clone, and gating strategy or; (b) >3% of events within the T, B and >5% on NK cells exhibiting OX40 signal above a positivity threshold set by a fluorescent-minus-one (FMO) control in the same assay of an age- and sex-matched healthy reference distribution measured on the same instrument, antibody clone, and gating strategy. In the method: The benefit of the treatment may be confirmed by determining that dendritic-cell OX40L or T, B or NK OX40 has decreased by ≥20% from a pretreatment value within 2-6 weeks after initiating therapy, the decrease being calculated using the same metric used to define OX40L or OX40 percent expression (percent-positive relative to FMO). The decrease may be ≥30%, ≥40%, or ≥50%, measured as cell-surface OX40L % on dendritic cells or OX40% on T, B or NK cells by flow cytometry.

In one of the above embodiments, administration of the drugs may be determined by: (i) maintaining the regimen if dendritic-cell OX40L decreases by ≥20% from baseline; (ii) increasing dose frequency (shorten interval) if OX40L decreases by <20% and no clinical response is present; (iii) discontinuing if OX40L increases by ≥10% and no clinical response is present; and (iv) maintaining the regimen despite an OX40L decrease <20% if a clinical response is present.

In the methods, particularly the diagnostic methods, the method can further comprising the steps of (a) contacting a portion of the sample with a candidate therapeutic agent (for example, adding an OX40L blocking antibody ex vivo during the culture) and (b) observing whether the agent suppresses the upregulation of OX40/OX40L, thereby predicting whether the patient would respond positively to the selected candidate therapeutic agent.

Another embodiment is directed to a method of reducing the risk of an acute FPIES during an oral food challenge or accidental ingestion, comprising (a) administering to the patient, a single dose of IL4R-a antagonist (such as dupilumab), OX40 or OX40L pathway inhibitor, and (b) exposing the patient to the food.

Another embodiment is directed to a kit comprising, (i) a vial of an IL-4Rα antagonist in a carrier and (ii) a vial of OX40 or OX40L pathway inhibitor in a pharmaceutically acceptable carrier. In the kit, the vial of the IL-4Rα antagonist may be provided in a first unit dosage form (such as a pre-filled syringe of dupilumab), and the OX40/OX40L pathway inhibitor may be provided in a second unit dosage form, packaged together with instructions that both agents are to be used as part of a combination therapy regimen for FPIES.

Another embodiment is directed to a diagnostic method for FPIES, comprising: (a) obtaining a biological sample containing immune cells from a patient (such as peripheral blood); (b) measuring immune cell (dendritic, T, B and NK cell) surface OX40 and OX40L by flow cytometry; (c) exposing the sample ex vivo to one or more food antigens suspected of causing FPIES; (d) measuring the expression of OX40 ligand (OX40L) by flow cytometry on dendritic cells in the sample, and/or the expression of OX40 on T, B or NK cells in the sample, and (e) comparing said expression to a baseline or control sample not exposed to the antigen.

Another embodiment is directed to a method of identifying a patient with FPIES who is likely to benefit from OX40/OX40L pathway inhibition, comprising; (a) performing an ex vivo T, B and NK cell activation assay on the patient's blood with the suspect food antigen, and (b) detecting the level of OX40+ cells with a predetermined threshold (3% for T and B cells and 5% for NK cells) and OX40L with a predetermined threshold of 2% above baseline.

Another embodiment is directed to a method of treating a human subject who meets Rome IV criteria for irritable bowel syndrome (IBS), the method comprising administering to the subject an effective amount of an IL-4 receptor-α (IL-4Rα) antagonist. In the method: The IBS may be food-triggered. The IL-4Rα antagonist may be a monoclonal antibody that binds IL-4Rα and blocks both IL-4 and IL-13 from signaling through said receptor. The IL-4Rα antagonist may be dupilumab, administered as a subcutaneous injection (for example, about 200-600 mg every two or four weeks in an adult or weight-adjusted equivalent in a child). The IL-4Rα antagonist may be dupilumab, administered as a subcutaneous injection at a dose of between 200-300 mg. The IL-4Rα antagonist may be rademikibart (also known as CBP-201), administered subcutaneously at a dose of about 150-300 mg every one to four weeks. The IL-4Rα antagonist may be manfidokimab (AK120), (for example, 200-300 mg every two weeks).

Another embodiment is directed to a method of treating IBS in a patient, comprising administering a therapeutically effective amount of an OX40 or OX40L pathway inhibitor. In the method: The OX40/OX40L pathway inhibitor may be a monoclonal antibody that binds OX40 to prevent its interaction with OX40L. The antibody against OX40 may be rocatinlimab (KHK4083/AMG 451), administered subcutaneously at a dose of about 150 mg to 600 mg every 2 or 4 weeks (with or without an initial loading dose). The antibody against OX40 may be telazorlimab (ISB 830), administered subcutaneously at a dose of about 300 mg every two weeks or 300 mg every four weeks (following a loading dose), or about 600 mg every two weeks. The OX40L pathway inhibitor may be a monoclonal antibody that binds OX40L (OX40 ligand) to prevent its interaction with OX40. The antibody against OX40L may be amlitelimab (KY1005), administered via subcutaneous injection at a dose of about 100 mg to 250 mg every four weeks (optionally with an initial loading dose of 200-500 mg).

In a method above, the patient may be administered both an IL-4Rα antagonist and an OX40 or OX40L pathway inhibitor (either concurrently as combination therapy, or sequentially within an overlapping treatment regimen). In the method: the IL-4Rα antagonist may be dupilumab, rademikibart or manfidokimab, the OX40 antagonist may be rocatinlimab, telazorlimab and the OX40L antagonist may be amlitelimab.

Another embodiment is directed to a method of inducing oral tolerance to a food allergen in a patient with IBS, comprising: (a) administering, to the patient a therapeutically effective amount of IL4a antagonist and (b) administering the food allergens in increasing amounts. In the method: Step (b) may comprise controlled exposure via an oral immunotherapy protocol or gradual dietary introduction and step (a) may comprise administering the IL-4Rα antagonist given biweekly throughout the protocol. Further, tolerance may be assessed by negative food challenge after at least 3-6 months of therapy. Also in the method: step (a) may be administering OX40 or OX40L pathway inhibitor

Another embodiment is directed to a method of determining whether an IBS patient would benefit from a IL4r-a antagonist, or OX40L/OX40 pathway inhibitor treatment by testing the patient's blood sample using flow cytometry to determine if: (a) ≥2% of events within either the CD11c⁺ myeloid dendritic-cell gate or the CD303 (BDCA-2)⁺ plasmacytoid dendritic-cell gate exhibiting OX40L signal above a positivity threshold set by a fluorescent-minus-one (FMO) control in the same assay of an age- and sex-matched healthy reference distribution measured on the same instrument, antibody clone, and gating strategy or; (b) >3% of events within either the T, B and >5% of events within the NK cells exhibiting OX40 signal above a positivity threshold set by a fluorescent-minus-one (FMO) control in the same assay of an age- and sex-matched healthy reference distribution measured on the same instrument, antibody clone, and gating strategy. In the method: The benefit of the treatment may be confirmed by determining that dendritic-cell OX40L or T, B or NK OX40 has decreased by ≥20% from a pretreatment value within 2-6 weeks after initiating therapy, the decrease being calculated using the same metric used to define OX40L or OX40 status (percent-positive relative to FMO). The decrease may be ≥30%, ≥40%, or ≥50%, measured as cell-surface OX40L % on dendritic cells or OX40 on T, B or NK cells by flow cytometry.

In the above methods: The administration of the drugs may be determined by: (i) maintaining the regimen if dendritic-cell OX40L decreases by ≥20% from baseline; (ii) increasing dose frequency (shorten interval) if OX40L decreases by <20% and no clinical response is present; (iii) discontinuing if OX40L increases by ≥10% and no clinical response is present; and (iv) maintaining the regimen despite an OX40L decrease <20% if a clinical response is present.

The above diagnostic method may further comprising the step of (a) contacting a portion of the sample with a candidate therapeutic agent (for example, adding an OX40L blocking antibody ex vivo during the culture) and (b) observing whether the agent suppresses the upregulation of OX40/OX40L or other activation markers, thereby predicting whether the patient would respond positively to the selected candidate therapeutic agent.

Another embodiment is directed to a method of reducing the risk of an acute IBS reaction during an oral food challenge or accidental ingestion, comprising (a) administering to the patient, a single dose of IL-4Rα antagonist (such as dupilumab), OX40 or OX40L pathway inhibitor, and (b) exposing the patient to the food.

Another embodiment is directed to a kit comprising, (i) a vial an IL-4Rα antagonist and (ii) a vial of OX40 or OX40L pathway inhibitor in a pharmaceutically acceptable carrier. In the kit: The vial of the IL-4Rα antagonist may be provided in a first unit dosage form (such as a pre-filled syringe of dupilumab) and the OX40/OX40L pathway inhibitor may be provided in a second unit dosage form, and they may be packaged together with instructions that both agents are to be used as part of a combination therapy regimen for IBS.

Another embodiment is directed to a diagnostic method for IBS, comprising: (a) obtaining a biological sample containing immune cells from a patient (such as peripheral blood); (b) measuring immune cell (dendritic, T, B and NK cell) surface OX40 and OX40L by flow cytometry; (c) exposing the sample ex vivo to one or more food antigens suspected of causing the patient's IBS; (d) measuring the expression of OX40 ligand (OX40L) by flow cytometry on dendritic cells in the sample, and/or the expression of OX40 on T, B or NK cells in the sample, and (e) comparing said expression to a baseline or control sample not exposed to the antigen.

Another embodiment is directed to a method of identifying a patient with IBS who is likely to benefit from OX40/OX40L pathway inhibition, comprising performing an ex vivo T, B and NK cell activation assay on the patient's blood with the suspect food antigen and detecting the level of OX40+ cells produced; wherein if the level exceeds a predetermined threshold (3% for T and B cells and 5% for NK cells), and OX40L with a threshold of 2% above baseline.

### BRIEF DESCRIPTION OF THE FIGURES

FIGS. 1A and 1B: Whole blood collected in heparin tubes was immune stained per the clinical standard immunophenotyping protocol (Amerimmune LLC, Fairfax, VA). In brief, for dendritic cell staining, 200 μl of whole blood was the following antibody combinations (Lineage consisting of CD3, CD14, CD16, CD19, CD20, CD56 and CD34− FITC), CD11C-PE, HLA-DR-PerCP eF710, CD303a-APC, and OX40L-PECy7 for 30 minutes at 4° C. To stain CRTH2 cells, 100 μl of whole blood was stained with the following antibody combination: CD4-FITC, CD45RO-PerCPCy5.5, CRTH2-APC, CD3-AF700, CD8-SB436 and CD45-EF506 for 30 minutes at 4° C. Cells were acquired on an ATTUNE NXT (ThermoFisher Scientific). FIG. 1A) Dendritic cells are identified as HLA-DR+Lineage-CD34− cells. DCs are separated into mDCs and pDCs as CD11c+CD303a− and CD11c−CD303a+, respectively. mDC and pDC OX40L+ cells were identified on OX40L/CD11c and OX40L/CD303a plots. FIG. 1B) T cells were identified as CD45+CD3+ cells. The CD4+CRTH2 and CD8+CRTH2 populations were subsequently identified using CRTH2 vs CD45RO plots gated on CD4+ cells and CD8+ cells, respectively.

FIG. 2: Peripheral blood mononuclear cells are incubated with PBS or wheat for 2 hours. Cells are then stained to identify myeloid (CD11c) and plasmacytoid (CD303a) dendritic cells. OX40L expression is shown on the x axis. Incubation with wheat (100 ng/mL) increases the mean fluorescence of OX40L an indication of increased number of OX40L molecules on the surface of cells

FIG. 3: Experiments were performed, and cell subsets were gated as described in FIGS. 1A and 1B. Percentages for each population—Tc2 cells (CRTH2⁺ CD8⁺ T cells), OX40L-positive plasmacytoid dendritic cells (pDCs), and OX40L-positive myeloid dendritic cells (mDCs)—were calculated relative to their respective parent populations.

FIGS. 4A and 4B: Representative flow cytometry scatter plot for normal OX40 and OX40L percentages on (FIG. 4A) T cells, CD4 T cells, CD8 T cells, B cells, NK, NKT cells and (FIG. 4B) Myeloid dendritic cells and Plasmacytoid dendritic cells. Fourteen samples were processed to determine normal percent values for OX40 on each cell type shown. Normal OX40 is approximately 2-3 percent for T cells and B cells respectively, and less than 5% for NK and NKT cells. Thirty-four samples were processed to determine normal percentage values for OX40L on myeloid and plasmacytoid dendritic cells. The normal OX40L percentage on both dendritic cells populations is approximately 2%. The threshold set by a fluorescent-minus-one (FMO) control in the same assay of an age- and sex-matched healthy reference distribution measured on the same instrument, antibody clone, and gating strategy.

### DETAILED DESCRIPTION

The present disclosure provides therapeutic, diagnostic, prognostic, and kit-based solutions for Food Protein-Induced Enterocolitis Syndrome (FPIES) and a Rome-defined irritable bowel syndrome (IBS) subtype, including food-triggered IBS. In one aspect, methods of treatment comprise administering an interleukin-4 receptor-alpha (IL-4Rα) antagonist—e.g., dupilumab—or, in alternative aspects, an inhibitor of the OX40/OX40L costimulatory pathway—e.g., anti-OX40 (rocatinlimab, telazorlimab) or anti-OX40L (amlitelimab). Certain embodiments encompass combination regimens where an IL-4Rα antagonist and an OX40/OX40L inhibitor are administered concurrently or sequentially. Dosing can be tailored within disclosed ranges (e.g., dupilumab about 200-600 mg every two or four weeks in adults or weight-adjusted equivalents in children; representative ranges for rademikibart, manfidokimab, rocatinlimab, telazorlimab, and amlitelimab are similarly provided). Co-morbid type-2 inflammatory conditions (e.g., atopic dermatitis, type-2 asthma, eosinophilic esophagitis, chronic rhinosinusitis with nasal polyps, chronic spontaneous urticaria, prurigo nodularis) are expressly contemplated.

In another aspect, the disclosure provides precision-medicine frameworks to identify and monitor responders using cell-surface biomarkers measured by flow cytometry. Illustrative selection criteria include: (i) ≥2% OX40L-positive events within CD11c⁺ myeloid dendritic cells and/or CD303⁺ plasmacytoid dendritic cells above an FMO-defined threshold; and/or (ii) >3% OX40-positive events on T or B cells and >5% on NK cells above an FMO-defined threshold, with thresholds established against age-/sex-matched healthy reference distributions using the same instrument, clones, and gating. In certain embodiments, clinical benefit is confirmed when dendritic-cell OX40L or lymphocyte OX40 decreases by ≥20% from pretreatment within about 2-6 weeks (with additional response bands at ≥30%, ≥40%, or ≥50%). The disclosure further teaches therapy-titration algorithms that (a) maintain dosing if OX40L decreases ≥20% or if there is clinical response despite a <20% decrease; (b) shorten dose intervals if OX40L decreases <20% without clinical response; and (c) discontinue if OX40L increases ≥10% in the absence of response.

Companion in-vitro and ex-vivo diagnostics are also provided. Representative methods include exposing patient blood to suspect food antigens and quantifying OX40L on dendritic cells and/or OX40 on T, B, and NK cells, optionally with ex-vivo addition of a candidate therapeutic (e.g., OX40L-blocking antibody) to assess suppression of OX40/OX40L upregulation and thereby predict in-vivo responsiveness. The disclosure further provides prophylactic and desensitization uses, including (i) reducing acute reaction risk during an oral food challenge or accidental exposure by administering a single dose of IL-4Rα and/or OX40/OX40L pathway inhibition prior to exposure; and (ii) inducing oral tolerance by administering IL-4Rα and/or OX40/OX40L inhibitors during controlled oral immunotherapy or graded dietary reintroduction, with tolerance confirmed by negative challenge after a defined interval (e.g., about 3-6 months).

Kit configurations are provided comprising unit-dose IL-4Rα antagonists and OX40/OX40L inhibitors, optionally co-packaged with instructions for combination use and with assay reagents to measure OX40/OX40L on defined immune-cell subsets. Collectively, these embodiments address unmet needs in FPIES and food-triggered IBS by coupling targeted immunomodulation with biomarker-guided selection, monitoring, and dose optimization to achieve remission, enable diet liberalization, mitigate challenge risk, and, in certain implementations, promote durable tolerance.

The subject invention relates to methods, compositions, and kits for treating Food Protein-Induced Enterocolitis Syndrome (FPIES) by modulating (i) the IL-4/IL-13→IL-4Rα signaling axis and/or (ii) the OX40-OX40L T-cell co-stimulation axis. While studying dendritic cell activation in immune disorders, it was found that the blood dendritic cells of patients with food induced gastrointestinal disease that is not IgE mediated, express high OX40L. This expression is further augmented with exposure to the offending foods, orally. The levels were much higher in FPIES, and food triggered IBD, compared to atopic dermatitis, asthma, chronic urticaria and others (Table 2). Furthermore, it was discovered that expression levels of OX40L and OX40 significantly declined in patients receiving dupilumab for various FDA approved indications of dupilumab, when these conditions co-existed with FPIES or food triggered IBS.

Contrary to its frequent association with Th2 polarization, OX40L expressed on antigen-presenting cells (e.g., dendritic cells) and functions as a lineage-agnostic co-stimulatory module that sustains and amplifies antigen-experienced T cells irrespective of their effector fate. Upon initial TCR priming, OX40 engagement delivers survival, proliferative, and anti-activation-induced cell death signals that (i) maintain and expand pre-committed Th programs (Th1 in IL-12-rich milieus; Th17 in IL-6/TGF-β milieus; Th2 in IL-4/IL-13 milieus), (ii) support T follicular helper (Tfh) cell persistence and IL-21 production that drive germinal center reactions and class switching, and (iii) augment CD8⁺ T-cell (Tc) effector and memory pools, including Tc1 and Tc2 subsets, depending on contextual cytokines.

In the setting of food protein-induced enterocolitis syndrome (FPIES), where mixed innate-adaptive signatures (Th2, Th1, Th17, Tc subsets, and impaired regulation) variably co-exist across patients and time, OX40L's lineage-agnostic role provides a coherent mechanistic rationale for targeting the OX40/OX40L axis. Inhibiting OX40 or OX40L is expected to attenuate pathologic T-cell survival, expansion, and recall regardless of whether Th2 cytokines predominate, thereby addressing heterogeneity that cannot be reliably predicted a priori. This property distinguishes OX40/OX40L blockade from pathway-specific cytokine, chemokine or receptor antagonists and supports its inclusion—alone or in combination—in biomarker-guided treatment algorithms for FPIES. OX40 (CD134) is a TNF-superfamily receptor expressed on activated T cells, especially T helper cells, CD8T cells, B and NK cells, while OX40L (CD252) is its ligand and has limited expression on antigen-presenting cells (APCs) such as dendritic cells, B cells, and others. OX40-OX40L engagement provides a potent second signal that promotes T-cell proliferation, survival, and differentiation.

The clinical biology of FPIES diverges from the skin/airway type-2 diseases as the closest food-allergy prior treatment strategy failed: a multicenter, open-label phase-II study of dupilumab monotherapy in peanut-allergic children—an archetypal IgE-mediated food allergy in which IL-4/IL-13 signaling drives class-switching—did not improve desensitization after 24 weeks on double-blind food challenge. It was discovered that OX40L is not elevated in IgE mediated food allergy either at baseline or on oral challenge with the offending food that results in systemic allergic reaction (anaphylaxis). By contrast, in this invention, human FPIES data demonstrate clinically meaningful remission accompanied by mechanistic shifts—notably reduction of OX40L on dendritic cells and modulation of Tc2—that have not been shown with dupilumab in its other approved indications and that map onto a costimulatory axis (OX40L-OX40) uniquely prominent in lesional tissue contexts different from gut FPIES (e.g., dendritic-cell OX40L in AD skin).

In various embodiments, the invention employs one or more antagonists of IL-4Rα and/or OX40/OX40L to reduce the severity, duration, and frequency of FPIES reactions, to enable food reintroduction, and to induce or maintain clinical tolerance. In certain embodiments, treatment is guided by biomarkers (e.g., OX40L expression on dendritic cells; OX40 expression on T, B and NK cells) measured ex vivo after relevant food antigen stimulation, and therapy is administered prophylactically, peri-exposure, acutely, and/or as maintenance.

Without being bound to theory, IL-4Rα blockade (e.g., dupilumab, rademikibart, manfidokimab) reduces Type-2 cytokine signaling, while OX40/OX40L antagonism (e.g., rocatinlimab, telazorlimab, amlitelimab) disrupts survival and expansion of antigen-experienced effector T cells; together they address heterogeneous, lineage-agnostic drivers of FPIES pathobiology.

### Definitions

As used herein, a “therapeutically effective amount” means an amount sufficient to achieve a clinically meaningful benefit (e.g., prevention or reduction of acute FPIES episodes, improvement of gastrointestinal symptoms such as diarrhea, bloating, pain, constipation and other symptoms, increased tolerated portion size, or induction/maintenance of food tolerance). For example, a loading dose of 600 mg dupilumab followed by 400, 300 or 200 mg every 1, 2, 3, 4 weeks or less frequent, or 400, 300 or 200 mg every 1, 2, 3 or 4 weeks without a loading dose is an exemplary regimen adaptable to FPIES; pediatric dosing may be weight tiered. “Patient” or “subject” refers to a human, including infants, children, adolescents, and adults with FPIES. Unless otherwise specified, ranges stated herein encompass all subranges and individual values. Where the term “about” is used, it denotes ±10% of the stated value, unless context dictates otherwise. The terms “comprising,” “including,” and “having” are open-ended and do not exclude additional elements or steps.

### Patient Selection and Biomarkers

In certain embodiments, therapy is selected and/or monitored using one or more biomarkers measured in whole blood or PBMCs stimulated ex vivo with culprit food antigens. Biomarkers include, without limitation: (i) increased percentage of OX40L on dendritic-cells and its subsets; (ii) OX40 on T, B and NK cells. Elevated OX40L/OX40 percentage compared above a specific baseline support the selection of an OX40/OX40L antagonist and/or an IL-4Rα antagonist for treatment. For OX40L, this is 2% above fluorescent minus one (FMO) on myeloid or plasmacytoid dendritic cells. For OX40, this is 3% above FMO on T cells, 3% on B cells and 5% on NK cells. The threshold was calculated based on age- and sex-matched healthy reference distribution measured on the same instrument, antibody clone, and gating strategy. FPIES has a unique biomarker expression profile compared to other atopic TH2 related disorders with the expression of OX40L on dendritic cells (Table 2).

TABLE 2

| | Myeloid | Plasmacytoid |
|---|---|---|
| N = 6/group | dendritic cells | dendritic cells |
| Asthma | 7.1% | 8% |
| Atopic dermatitis (AD) | 4.1% | 4.2% |
| CRS with nasal polyps | 3.2% | 2.5% |
| (CRSwNP) | | |
| CIU | 0.9% | 1% |
| FPIES | 15% | 22% |
| FPIES + Asthma | 22.5% | 29% |
| FPIES + AD | 24% | 29% |
| FPIES + CRS | 14 | 21% |
| FPIES + CIU | 13% | 19.2% |
| Food triggered IBS | 23% | 26% |
| Non-food triggered IBS | 2.1% | 1.6% |

Table 2: OX40L expression on DCs in various allergic and immune conditions: Average percentage of dendritic cell subsets expressing OX40L in various disease states, including when these diseases co-exist. FPIES exhibits markedly elevated OX40L expression on both dendritic cell subsets, even in the presence of other TH2 comorbidities. OX40L overexpression appears to be a distinctive feature of FPIES. The numbers are average expression percentage of OX40L on dendritic cell subsets (myeloid or plasmacytoid). The co-existence of FPIES with other conditions raises the OX40L expression further, suggesting a unique mechanism when present together. This may not indicate an additive effect. These results indicate a complex relationship between these different allergic conditions further supporting the non-obviousness of the IL4ra, OX40L and OX40 inhibition approaches in FPIES and IBS.

### METHODS OF TREATMENT

The methods of the invention are useful for treating acute, chronic, or atypical FPIES in pediatric and adult patients reacting to one or multiple foods (e.g., cow's milk, soy, rice, oats, wheat, barley, fish, shellfish, poultry, legumes and all other relevant food allergens). Treatment may be delivered (a) preventatively/prophylactically before expected food exposure (e.g., prior to supervised oral food challenges or at home reintroduction), (b) at the onset of symptoms to blunt progression, and/or (c) as chronic maintenance to prevent episodes and enable diet liberalization and (d) tapered over time as tolerance while on the treatment is achieved.

### Therapeutic Agents of the Invention

IL-4Rα antagonists: Exemplary agents include dupilumab, rademikibart (CBP-201), and manfidokimab (AK120). In adults, dupilumab may be given as a 600 mg loading dose followed by 200-400 mg every 4 weeks (Q4W), 3 weeks (Q3W), 2 weeks (Q2W) or weekly (Q1W) or less frequently. In pediatric subjects, weight-tiered regimens are used; in certain embodiments, 200 mg every 4 weeks (Q4W) is administered (e.g., infants/toddlers), with adjustment based on clinical response and body mass. Rademikibart regimens include 150-300 mg Q2W, with monthly (Q4W) dosing contemplated for maintenance. Manfidokimab may be administered up to ~300 mg Q2W in certain embodiments. Rademikibart and manfidokimab, can alternatively be dosed like dupilumab as described above.

OX40/OX40L antagonists: Exemplary agents include rocatinlimab (anti-OX40), telazorlimab (anti-OX40), and amlitelimab (anti-OX40L). In certain embodiments, rocatinlimab is administered at 150-600 mg Q4W-Q1W; telazorlimab at 150-600 mg Q4W-Q1W (with or without a loading dose); and amlitelimab at 150-600 mg Q4W-Q1W (with or without a loading dose). Doses may be given subcutaneously (preferred) or intravenously (for vial/lyophilized presentations). These OX40/OX40L inhibitors can alternatively be dosed like dupilumab as described above.

Combination regimens: In some embodiments, an IL-4Rα antagonist and an OX40/OX40L antagonist are co-administered (concurrently or sequentially) to provide complementary suppression of soluble cytokine signaling and effector T-cell survival/recall. For example, dupilumab Q2W may be paired with rocatinlimab Q4W for induction, followed by monotherapy maintenance.

### Dosing Schedules and Algorithms

In one algorithm, a patient begins with induction (e.g., dupilumab 600 mg load→300 mg Q2W OR Q1W×4-8 weeks; or rocatinlimab 300-600 mg Q2W×8-12 weeks), followed by response-based maintenance (e.g., Q2W or Q4W). Food reintroductions occur after an initial response window (e.g., days to weeks) under supervision; successful introductions are consolidated with continued dosing, then tapered per biomarker and symptom stability (e.g., extension to Q4W, then trial discontinuation with monitoring). Acute exposures/accidents may be managed by on-schedule dosing or an on-demand injection per clinician protocol.

### Pharmaceutical Compositions

Pharmaceutical compositions comprise a therapeutically effective amount of one or more active agents selected from IL-4Rα antagonists and OX40/OX40L antagonists in a pharmaceutically acceptable carrier suitable for the intended route (e.g., subcutaneous injection, IV infusion). Single-dose presentations are advantageous for adherence and dosing accuracy. The concentration of active ingredient is selected considering absorption/clearance, physicochemical properties, dosing schedule, and the desired serum exposure.

In particular embodiments, unit dosages contain, for example, about 75 mg, 150 mg, 200 mg, 300 mg, or 600 mg of antibody in 0.5-3.0 mL for SC administration (pre-filled syringe or autoinjector). More generally, unit dosage strengths of about 1-1000 mg are contemplated, including 5-250 mg and 10-50 mg intervals for smaller presentations; unit doses may be adjusted for pediatric weights.

### Formulation

Compositions may include buffers (e.g., histidine, citrate), tonicity agents, stabilizers (e.g., trehalose, sucrose), surfactants (e.g., polysorbate), and other excipients known to maintain stability, reduce aggregation, and ensure syringeability. Formulations may be aqueous solutions for SC injection or lyophilized powders for reconstitution (IV infusion). Target pH typically ranges from about 5.0-6.5 for antibody stability; isotonicity is preferred. The therapeutically effective concentration is determined empirically and translated to human dosing.

### Unit Dosage Forms and Serum Exposure

In some embodiments, serum concentrations achieved by the dosing regimens described herein fall within ranges sufficient to maintain target occupancy and clinical effect, e.g., from about ng/ml to tens of μg/mL, depending on agent and interval. Dosing can be titrated to maintain troughs within a predefined window while minimizing adverse events.

### Routes of Administration

Subcutaneous administration is preferred for antibodies; intravenous infusion is used for lyophilized/vial presentations when indicated. The invention also contemplates intradermal and other routes compatible with biologic delivery, where appropriate.

### Articles of Manufacture

Articles of manufacture include packaging material, a pre-measured unit (e.g., pre-filled syringe or autoinjector containing 150-300 mg/1-2 mL), and labeling indicating use for treating or preventing FPIES or its symptoms. Packaging may include blister packs, cartons, vials, syringes, or pens, with instructions for storage (e.g., 2-8° C.; protect from light; do not freeze) and handling.

### Kits

Kits may include: (i) one or more-unit doses of an IL-4Rα antagonist and/or an OX40/OX40L antagonist; (ii) instructions for dosing schedules (induction, maintenance, taper, acute management); and, optionally, (iii) companion diagnostic reagents for ex vivo stimulation and flow cytometry of OX40L/OX40. Components may be provided pre-loaded or separately with a diluent; instructions may be physical or electronic.

### Combination Therapies

Combination therapy refers to regimens where two agents are given in temporal or biochemical relationship (e.g., concurrent dosing; sequential dosing keyed to troughs or clinical milestones). Antihistamines, antiemetics (e.g., ondansetron), IV fluids, or oral rehydration may be co-administered as supportive care without departing from the invention.

### Treatment Paradigms

### Prophylaxis and Induction

In one embodiment, patients with frequent episodes or planned food challenges receive induction with an IL-4Rα antagonist (e.g., dupilumab 600 mg load→300 mg Q2W/Q1W) and, where indicated by biomarkers (e.g., OX40L-high DCs), an OX40/OX40L antagonist (e.g., rocatinlimab 300-600 mg Q2W or amlitelimab 250 mg Q4W). Food reintroduction begins after initial symptom reduction (often days to weeks) under supervision.

### Maintenance and Taper

Upon achieving stability (e.g., absence of reactions with routine exposures; negative/normalized biomarker panel), dose intervals may be extended (e.g., Q4W) and ultimately discontinued with post-discontinuation monitoring and predefined criteria for retreatment.

### Timing of Treatment

The initiation of dupilumab therapy in food protein-induced enterocolitis syndrome (FPIES) is a critical determinant of its clinical effectiveness.

In some embodiments, administration of dupilumab is initiated following confirmation of a positive food challenge, during the acute phase of disease, to prevent recurrence of delayed gastrointestinal symptoms. Early initiation during or shortly after a confirmed episode may dampen the FPIES symptoms, preventing progression to chronic gastrointestinal inflammation and improving tolerance development. In other embodiments, dupilumab therapy is begun in the remission phase, between symptomatic episodes, with the goal of preventing recurrence upon re-exposure to the trigger food. Such an approach is designed to stabilize immune pathways before the next antigen encounter. In some examples, treatment with IL4Ra, OX40L and/or OX40 inhibitor can be administered to human subjects with chronic food induced gastrointestinal symptoms that is atypical FPIES, that overlaps with irritable bowel syndrome (IBS) that is triggered by food ingestion.

Timing of dosing may also follow a loading and maintenance strategy, wherein a loading dose is administered within hours to days after an acute FPIES reaction, followed by regularly scheduled maintenance doses (e.g., weekly or biweekly). This regimen ensures immediate receptor saturation to control acute immune dysregulation, with subsequent dosing maintaining blockade of IL-4/IL-13 signaling during the high-risk period for re-exposure.

Additionally, dupilumab therapy may be optimized around planned food reintroductions. In certain embodiments, patients are administered dupilumab for a defined induction period (e.g., 4-12 weeks) prior to supervised oral food challenges. This strategy reduces the likelihood of severe reactions during diagnostic or therapeutic re-exposures. In other embodiments, dupilumab is continued after tolerance induction to sustain long-term remission and reduce the risk of relapse.

Taken together, the timing of dupilumab initiation—whether at the acute phase, during remission, or in anticipation of food reintroduction—may significantly influence therapeutic outcomes in FPIES. Tailoring therapy to the clinical stage provides opportunities to both reduce immediate morbidity and potentially alter the long-term natural history of the disease.

### EXAMPLES

### Example 1

An 18-month-old male patient presents with multiple food allergies causing FPIES. Symptoms are vomiting to multiple food allergies; peach, milk, soy, oat, barley, tree nuts, fish (white fish) and rice. There is no IgE mediated reactions as demonstrated with negative skin testing and absent serum specific IgE levels to these foods. Patient has a total IgE level of 6 kU/L. The patient has eczema covering 75% of his body. The patient is treated with 200 mg dupilumab, monthly. The eczema started improving in 2 months-time. Significant improvement in all gastrointestinal symptoms was observed within days (3 days) of initiating therapy. Upon introduction of the offending foods described above, patient started to be able to tolerate them with no gastrointestinal symptoms was observed within 3 days of initiating therapy. Initially rice was introduced (on day 3 post dupilumab administration), followed by 6 days later oat challenge in the office and then 2 weeks later barley and milk challenge in the doctor's office on separate days. The remaining foods were introduced subsequently 1-2 months later with no gastrointestinal reactions.

### Example 2

A 5-year-old female patient with multiple food allergies (oat, milk and soy) with no IgE mediated sensitivity on skin testing resulting in FPIES (vomiting and diarrhea 2-3 hours and 6 hours respectively after the ingesting of the foods). The patient received 200 mg of dupilumab monthly, leading to a marked reduction in the severity and frequency of FPIES episodes and symptoms. Food introduction was initiated 15 days after the start of therapy, initially oat, followed several days later with milk and then soy the following month). The patient was on dupilumab for 6 months and then stopped. Patient continued to tolerate the offending foods.

### Example 3

A 55-year-old adult with wheat-induced enterocolitis syndrome. Immune evaluation showed OX40L on myeloid and plasmacytoid dendritic cells to be 16% and 22% respectively. T cell OX40 was 18%. His allergy testing to wheat was negative. The patient was treated with 300 mg of dupilumab every other week. Rapid improvement in FPIES, gastrointestinal symptoms was noted within days of starting the therapy. The symptoms that improved were diarrhea and abdominal bloating/pain. The dupilumab was continued every 2 weeks and patient continued to tolerate wheat.

### Example 4

A 52-year-old man was referred to our tertiary allergy-immunology clinic for evaluation of eczema and wheat-associated lower-gastrointestinal symptoms that had been labelled “ulcerative colitis” nearly two decades earlier. Born in France, he emigrated to the United States at age twenty-one to practice as a chiropractor. His medical history was unremarkable until age twenty-eight, when he experienced abrupt crampy abdominal pain followed one to three hours later by profuse watery diarrhea. Each episode comprised four to eight large-volume stools over twelve hours and was notable for visible blood and mucus. Attacks were invariably precipitated by wheat-containing foods—bread, pasta, pastries—and were never accompanied by urticaria, angioedema, wheeze, pruritus, or oropharyngeal tingling. Between attacks he felt entirely well.

An allergy evaluation at age thirty, including skin-prick testing and serum specific IgE to food and environmental panels, was negative. At age thirty-two, a severe flare necessitated emergency-department care for dehydration. Laboratory studies showed normal hemoglobin, slight elevated white-cell count with neutrophilia, C-reactive protein, erythrocyte sedimentation rate, and serum IgE. Colonoscopy revealed mild, patchy erythema of the sigmoid and descending colon. Histology demonstrated focal cryptitis, mild architectural distortion, and a modest increase in lamina-propria eosinophils (<20 per high-power field) without basal plasmacytosis, granulomas, or dysplasia—findings consistent with food-driven injury. Mesalamine was prescribed empirically, but remission occurred only after a strict wheat-elimination diet was initiated. Upper endoscopy was normal.

During the next decade he maintained gluten avoidance with excellent control. Four accidental open challenges—restaurant soy sauce, craft beer, French beignets, and home-cooked pasta—each provoked identical attacks within four hours, whereas minor cross-contact (e.g., a shared toaster) was tolerated, indicating a threshold effect rather than extreme sensitization.

At age fifty he developed pruritic, lichenified eczematous plaques on the antecubital fossae and neck that failed high-potency topical corticosteroids. Baseline assessment showed a height 178 cm, weight 79 kg (BMI 25 kg m⁻²), blood pressure 110/78 mm Hg, pulse 68 min⁻¹, temperature 36.6° C. SCORAD (Scoring Atopic Dermatitis) was thirty (moderate disease). Topical high potency steroid use only provided temporary relief. Abdominal exam was benign. Laboratory profile (Table 3) showed normal complete blood count, metabolic panel, serum IgE, and tissue-transglutaminase IgA. Allergy evaluation for environmental and food allergy was negative with skin prick testing. He did not have eosinophilia in on his complete blood count. Immune evaluation showed normal T, B, NK counts, however, there was an increase in CD8-T cell caspase-1 expression and CD8 effector memory T-cells. There was no family history of celiac disease, inflammatory bowel disease, or eosinophilic gastrointestinal disease; a half-sister reported intermittent mild hematochezia of unclear etiology.

TABLE 3
Patient Characteristics

| | | |
|---|---|---|
| | | Reference |
| Test (units) | Result | range |

Complete blood count

| | | |
|---|---|---|
| White cell count (×10³/μL) | 4.9 | 3.4-10.8 |
| Hemoglobin (g/dL) | 13.7 | 13.0-17.7 |
| Platelets (×10³/μL) | 262 | 150-450 |
| Absolute neutrophils (×10³/μL) | 2.9 | 1.4-7.0 |
| Absolute lymphocytes (×10³/μL) | 1.4 | 0.7-3.1 |

Basic metabolic panel

| | | |
|---|---|---|
| Creatinine (mg/dL) | 1.04 | 0.76-1.27 |
| BUN (mg/dL) | 15 | 6-24 |
| Calcium (mg/dL) | 9.6 | 8.7-10.2 |
| AST/ALT (IU/L) | 16/15 | 0-40/0-44 |

Inflammatory markers

| | | |
|---|---|---|
| C-reactive protein (mg/L) | 4 | 0-10 |
| Erythrocyte sedimentation | 8 | 0-30 |
| rate (mm/hr) | | |

Autoantibodies

| | | |
|---|---|---|
| ANA titer, pattern | 1:40, speckled | <1:40 |

Immunoglobulins

| | | |
|---|---|---|
| IgG (mg/dL) | 1108 | 603-1613 |
| IgA (mg/dL) | 269 | 90-386 |
| IgM (mg/dL) | 132 | 20-172 |
| Total IgE (IU/mL) | 12 | 6-495 |

Allergen-specific IgE

| | | |
|---|---|---|
| Wheat IgE (kU/L) | <0.10 | <0.10 |

Complement

| | | |
|---|---|---|
| C3 (mg/dL) | 116 | 82-167 |
| C4 (mg/dL) | 29 | 14-44 |

Flow-cytometric immunophenotyping (selected abnormalities)

| | | |
|---|---|---|
| Activated caspase-1 in CD8⁺ cells (%) | 39.5 | <10 |
| CD8⁺ Tem (CD45RO⁺CCR7⁻) (%) | 73.2 | 11.0-53.7 |

As a therapeutic intervention, dupilumab 300 mg subcutaneously every two weeks was started to manage his atopic dermatitis. After only two injections he accidentally ingested a wheat baguette while travelling in France and, for the first time in ten years, developed no gastrointestinal reaction. Encouraged, we conducted three further open challenges, each involving 3-4 days of escalating wheat ingestion, starting with a cumulative daily dose of ≈15 g of wheat protein, up to 50 g in the form of croissants, wheat bread sandwich, pancakes and baguette. All challenges were uneventful.

Six months later, insurance changes forced discontinuation of dupilumab. Within eight weeks he experienced a typical flare after eating homemade pizza abroad, characterized by crampy pain and bloody diarrhea. Endoscopy showed inflammation but absent eosinophils and no cryptitis. The attack responded promptly to a two-week taper of oral prednisone (40 mg daily for five days, then gradual reduction). Upon re-establishing coverage, dupilumab was resumed; after two doses he passed a supervised wheat challenge involving commercially prepared pasta, remaining symptom-free during 48 hours of monitoring. This observation proves that the patient did not outgrow the wheat related FPIES.

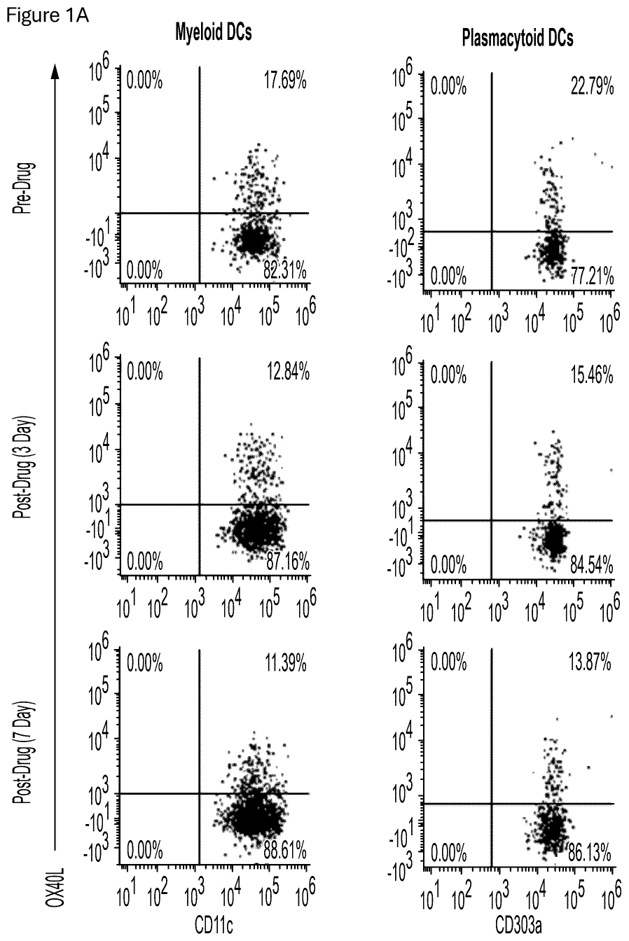

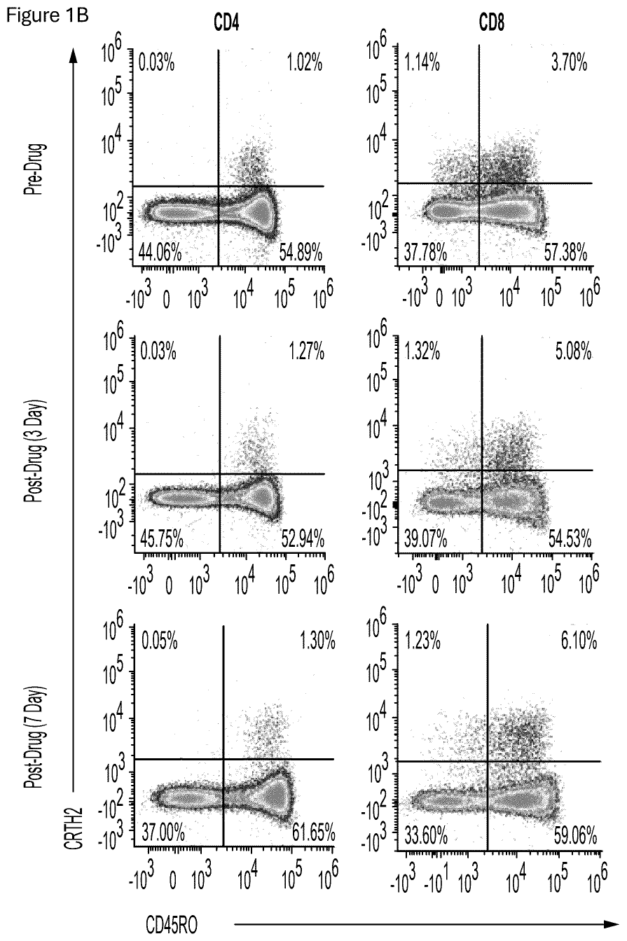

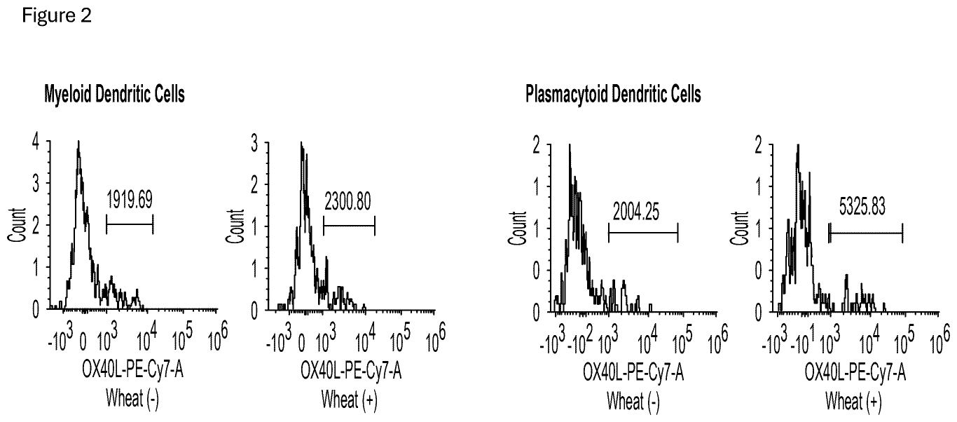

Serial flow cytometry at the time of re-initiating dupilumab, showed reduced OX40L-expressing dendritic cells from 22% at baseline to 12% within one week. Conversely, CRTH2-positive T cells (Chemoattractant receptor-homologous molecule expressed on Th2 cells—CRTH2) rose from 4% to ~10% of the CD8⁺ (Tc2) compartment, without any significant changes in CRTH2 expression on CD4⁺ Th2 cells. The OX40L and Tc2 stabilized around 8-10% and 12% respectively at 8-weeks' time. Pre-therapy, whole-blood stimulation with wheat extract induced a 1.2-fold increase in OX40L mean-fluorescence intensity in myeloid dendritic cells and 2.6-fold increase in plasmacytoid dendritic cells, confirming dendritic-cell responsiveness to the allergen. This wheat induce OX40L upregulation is not observed in healthy subjects. (FIGS. 1 and 2).

### Additional FPIES Examples as Shown in Table 4

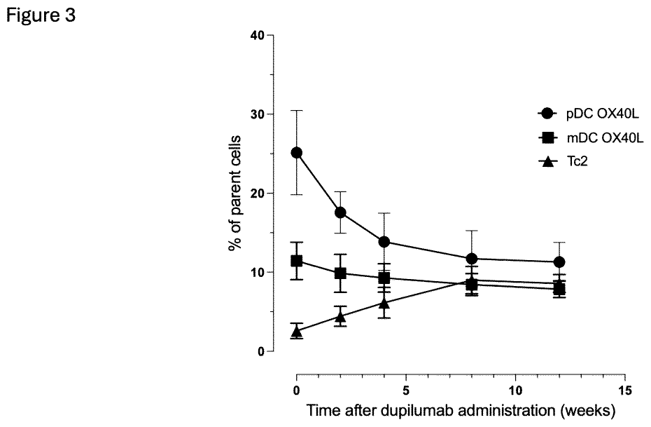

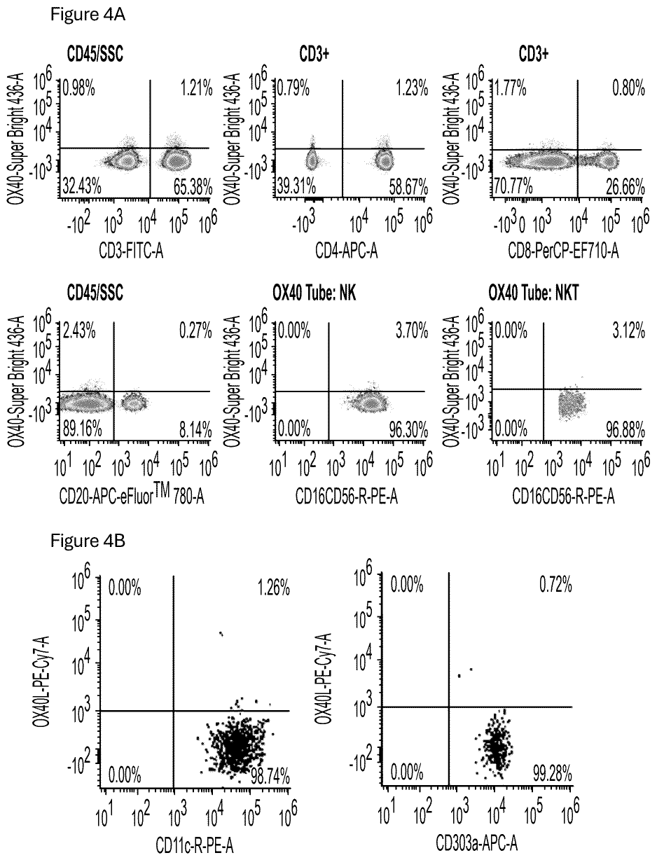

The invention further describes 7 additional patients with FPIES, who were started on dupilumab therapy, based on several FDA approved co-morbid conditions (Table 4). On clinical follow up all patients were able to tolerate all the foods they had significant gastrointestinal reactions to, despite wide variability in triggers (cow's milk, soy, rice, wheat, and shellfish) and disease chronicity (6 months-18 years). The resolution in gastrointestinal symptoms were complete. The decrease in plasmacytoid dendritic cell OX40L along with expansion of circulating CRTH2+ Tc2 cells followed the pattern observed in the original case report (FIG. 3). No serious adverse events occurred in any of the patients. These data underscore both the safety and the broad applicability of IL-4Rα blockade for FPIES remission across a wide age range.

TABLE 4

| Age | Indication | Foods | Symptom | IgE | Dose | Time to full remission |
|---|---|---|---|---|---|---|
| 18 M; | Eczema | Milk, soy, | Vomiting in 2 | 2 | 200 | All foods were |
| F | | egg, all | hours | | q4w | reintroduced in a 3-week |
| | | grains | | | | period without any |
| | | | | | | symptoms |
| 38; Y | Prurigo | Multiple | Diarrhea and | 10 | 300 | All foods reintroduced in |
| | Nodularis | (>10) | bloating | | q2w | 2-month period. Patient |
| | | | lasting 2-3 | | | stated gaining so much |
| | | | days | | | weight that he had to start |
| | | | | | | Ozempic. |
| 4 Y; M | Eczema | Milk | Vomiting | 5 | 300 | Milk reintroduced in 2 |
| | | | | | q2w | weeks |
| 30 Y; | Asthma | Milk, | Diarrhea, | 18 | 300 | Foods introduced in the |
| M | | Soy, | bloody stool | | q2w | order of milk, soy and |
| | | Wheat | | | | wheat over a course of 4 |
| | | | | | | weeks |
| 28 Y; | Nasal | Shellfish, | Vomiting, | 122 | 300 | Passed multi food |
| F | polyps | Fish | stomach pain | | q2w | challenge in 2 weeks after |
| | | | | | | therapy |
| 2; M | Eczema | Milk | Vomiting in 1 | 19 | 200 | Passed milk challenge in 4 |
| | | | hour | | q4 | weeks after therapy |
| 68; F | Asthma | Walnut, | Diarrhea, | 10 | 300 | Passed challenge in 3 |
| | | cashew, | colitis | | q2 | months. |
| | | pistachio | | | | |

### Table 4: FPIES Examples

Age is shown in years (y) or months (m); sex is indicated as F (female) or M (male). “Foods” lists the culprit allergens provoking FPIES reactions before treatment. “Symptom” records the predominant acute manifestation at the time of reaction. IgE is the total serum immunoglobulin E concentration, reported in international units per liter (IU/L). None of the patients showed any evidence of IgE mediated food allergy based on skin prick testing and serum specific IgE titers. All patients had confirmed medical history of reactivity to each food and resolution of symptoms once excluded from the diet. For pediatric patients, there was at least 6 months from the time of initial diagnosis and 1 month from last symptoms. For adult patients this was 20 years and 3 months respectively. No patient, based on clinical findings had naturally outgrown FPIES at the time of initiation of dupilumab therapy for their co-morbid disease. Dose refers to the subcutaneous dupilumab loading/maintenance regimen, expressed as milligrams (mg) followed by dosing interval: q2w=once every 2 weeks; q4w=once every 4 weeks. “Time to full remission” indicates the interval from first dupilumab dose to complete, sustained re-introduction of all offending foods without recurrence of symptoms, as documented by oral food challenges or dietary history.

Abbreviations: FPIES, food-protein-induced enterocolitis syndrome; IgE, immunoglobulin E; q2w, every 2 weeks; q4w, every 4 weeks.

### Example 5

26-year-old man, BMI 23.1 kg/m², Symptoms (18 months): Post-prandial cramping 30-90 min after meals, alternating stool form (Bristol 2-6), episodic delayed emesis at 2-4 h specifically after wheat and milk. Daily bloating, urgency during loose-stool weeks. No nocturnal diarrhea, weight loss, fevers, or GI bleeding. Comorbidities: Moderate persistent asthma, controlled on high-dose inhaled steroids; ACT 17 (not well-controlled). No atopic dermatitis; intermittent rhinitis. Medications/Trials pre-referral: Fiber (worse gas), peppermint oil (no effect), rifaximin 550 mg TID×14 d (transient 10-15% improvement), food avoidance, (partial, inconsistent), loperamide PRN. Rome IV: Meets criteria for IBS-M (>6 months; weekly pain related to defecation, change in stool frequency and form). Food trigger documentation: 4-week digital diet-symptom diary with lagged cross-correlation: wheat and milk associate with pain/loose stools at +2-6 h, but not with other foods. Open graded challenges (separate days): wheat (20→40→60 g flour equivalents) and milk (250→500 mL) each reproduce cramping and emesis at 2-4 h with stable vitals. To Rule-out organic disease, laboratory evaluation was normal. Celiac was negative by blood test and endoscopy. Fecal calprotectin 24 μg/g (repeat 31 μg/g). Stool PCR panel ×2 negative; fecal elastase 468 μg/g. Breath tests: lactulose/glucose negative for SIBO; lactose/fructose malabsorption negative. Colonoscopy with random biopsies incl. TI: normal; no microscopic colitis or eosinophilic colitis. Asthma baseline: Spirometry: Forced expiratory Volume FEV1 76% predicted, FEV1/FVC 0.72. FeNO (exhaled nitric oxide): 38 ppb (parts per billion) (elevated). Flow cytometry showed OX40L+mDC (CD11c⁺) to be 18.4%, and OX40L+pDC (CD303⁺) to be 19% above FMO. T-cell: CRTH2⁺ Tc2 6.6% of CD8 (ref 0.8-1.5%). Ex-vivo stimulation (6 h, wheat at 100 ng/mL): mDC OX40L→26%, pDC→27.9%. Interpretation: Th2/OX40L-high, food-reactive DC phenotype concordant with clinical wheat/milk triggers and coexisting T2-asthma. Indication chosen: Asthma (on-label). Hypothesis: IL-4Rα blockade will down-modulate OX40L on DCs and reduce IBS symptoms triggered by wheat/milk. Regimen: Dupilumab 600 mg SC load→300 mg Q2W.

Patient demonstrated rapid and durable clinical improvement by Week 12; narrowed stool-form; the Bristol profile tightened from a wide “IBS-M” spread (Types 2-6) to a stable, mostly Type 4 pattern after dupilumab. Elimination of symptoms on wheat challenge concordant with a stepwise reduction in OX40L (>20%). There was concomitant asthma improvement supports on-target IL-4/IL-13 pathway inhibition. Patients T cell OX40 also went down from pre-treatment value of 19% to 8% week 6 of therapy with dupilumab.

### Example 6

A 34-year-old woman, research analyst, BMI 22.3 kg/m². Chief complaint: Daily abdominal cramping with urgent loose stools for 18 months. Symptom profile (preceding 3 months): Abdominal pain: 4-6/10 most days, worse mornings. Bowel frequency: 3-5/day, Bristol 6-7 on ≥70% of days. Prominent urgency; occasional fecal incontinence (2×/month). Bloating daily; no nocturnal diarrhea; no reproducible food triggers (see diaries, challenges below). Exacerbated by psychosocial stress and sleep deprivation; menses has mild effect (looser stools day-1 to +2). Negative features: No weight loss, fevers, hematochezia/melena (rare streaks only with hemorrhoids), no extra-intestinal inflammatory symptoms, no atopic dermatitis, asthma, or chronic rhinosinusitis with polyps. Seasonal allergic rhinitis in childhood only. Medications tried before referral: Loperamide PRN (partial, inconsistent), rifaximin 550 mg TID×14 d (no benefit), low-dose amitriptyline 10-20 mg qHS (stopped for morning grogginess), peppermint oil (no change), fiber (worse bloating), mindfulness course (modest coping, no stool change). No bile acid sequestrants used prior to evaluation. These place her IBS-D as per Rome classification. Food trigger assessment: 4-week blinded food/symptom diary with time-stamped entries and automated cross-correlation analysis: no significant associations between symptoms and specific foods, including wheat, dairy, egg, soy, nuts, caffeine, or alcohol. Open titration challenges (milk 12-24 g lactose equivalent with lactase cover and without; wheat 16-32 g; soy 25 g; egg 2 whole equivalents) separated by ≥48 h: no reproducible symptom provocation beyond background variability. Laboratory & stool testing overall normal, negative celiac scree, no positive allergies on food allergy testing, normal upper and lower endoscopy as well as celiac panel. Breath test negative. MR enterography normal. Total IgE: 42 IU/mL (normal). Flow cytometry (peripheral blood): CD11c⁺ myeloid DC OX40L⁺: 0.9% above FMO (lab's “high” threshold ≥2.0% or ≥95th percentile). CD303⁺ plasmacytoid DC OX40L⁺: 0.3% above FMO. CD8⁺CRTH2⁺ Tc2: 1.2% of CD8 T cells (reference 0.8-1.5%). CD4⁺CRTH2⁺ Th2: 1.6% of CD4 T cells (ref 1-2%). CD3+T cell OX40: 1.2%. Interpretation: Type-2/OX40L-low endotype at baseline. Prior Management (pre-biologic): Targeted antidiarrheals: Scheduled loperamide 2 mg BID with PRN doses→modest urgency reduction, no change in pain. Rifaximin re-trial 550 mg TID×14 d→no change. Eluxadoline 75 mg BID (no gallbladder history)×6 weeks→improved urgency, but persistent pain and loose stools; stopped due to nausea. Bile acid sequestrant trial (colesevelam 625 mg 1-2 tabs BID) despite normal bile acid testing→minimal effect; discontinued. Neuromodulation: Nortriptyline 10→25 mg qHS×6 weeks→minor pain relief; stopped for dry mouth/sedation.

Despite the OX40L-low phenotype and lack of atopy, the patient elected to try dupilumab after informed consent, based on co-existing chronic urticaria. Received 600 mg s.c. loading, then 300 mg every 2 weeks. Duration: 16 weeks. Hives resolved by 80% 1 week after the loading dose. IBS-D related symptoms exhibited no clinical response to dupilumab; no change in Bristol mean, urgency episodes, abdominal pain or bloating. There was no change in the OX40L or OX40 expression on immune cells at 16 weeks. No side effect to the dupilumab. Patient continued the dupilumab given the significant benefit on the chronic urticaria. The case supports an inference that IBS patients without a food trigger or a OX40L/OX40 signal are unlikely to benefit from IL-4Rα blockade.

### Summary of the Examples

Prior dupilumab literature does not show OX40L down-modulation or Tc2 normalization, yet the FPIES and food triggered IBS patients demonstrate significant OX40L reduction and/or Tc2 shift with correlated clinical remission/tolerance. Furthermore, the magnitude (full remission of FPIES), rapidity and durability, of the clinical improvement in FPIES is different than the partial improvements seen in all other dupilumab indications.

Since FPIES is mast-cell-independent and largely T-cell/innate with unclear type-2 cytokine leverage, the field does not teach that IL-4Rα blockade would normalize OX40L/OX40 or induce tolerance—indeed, the absence of those signals in AD/asthma/EoE would discourage expecting it.

The demonstration of peripheral blood dendritic cell OX40L level change and Tc2 frequency/function are predictive for response in FPIES (biomarker-clinical linkage).

Taken together—(i) the negative peanut-allergy trial with the same biologic, (ii) the distinct, non-IgE, mast-cell-independent and multifactorial cytokine/chemokine profile of FPIES pathophysiology, and (iv) the unique biomarker-linked clinical response (OX40L and Tc2 cells) and (v) absent Th2 cell changes on dupilumab therapy—provide compelling evidence of the unique nature of mechanism of action of dupilumab in FPIES through a mechanism that leads to blunting of dendritic cell OX40L. Moreover, the combination of targeting both IL-4Rα and OX40/OX40L pathways, or selecting between them based on patient biomarkers, is unique to this invention.

In summary, there remains a need for an effective treatment for FPIES that goes beyond avoidance and supportive care. The prior art points to the general importance of IL-4/IL-13 in allergic disease and the OX40/OX40L axis in T-cell driven allergic inflammation, but it does not teach, mention, discuss or propose how to apply these to FPIES. The present invention fills this gap by providing targeted immunotherapies (anti-IL-4Rα and anti-OX40/OX40L biologics) to intercept the pathological immune response in FPIES, resulting in clinical improvement of one or more symptoms of FPIES. It teaches dosing amount, frequency, and efficacy in different clinical situations as described in the real-life cases. Additionally, this invention provides companion diagnostic methods using biomarkers such as OX40L and CRTH2 to identify patients who would benefit from such therapies and to monitor their response, further distinguishing it from prior approaches.

Further mechanistic observations, as shown in Table 3, demonstrate that peripheral blood dendritic cells over-expressing OX40L is selectively elevated in patients with FPIES compared to other TH2-related diseases, where the OX40L expression data are lesional/at the tissue level rather than in the blood. This indicates that FPIES has a different mechanism of action compared to the other TH2 disorders and that one cannot predict the response based on experience with the other TH2 conditions. In diseases where OX40L is not elevated on its own, its levels further increase when the disease occurs as a co-morbid condition with FPIES. This is potentially due to the impact of the FPIES immune response on the immune response of other conditions.

### REFERENCES

1. Nowak-Wegrzyn A, et al. Food protein-induced enterocolitis syndrome: guidelines for diagnosis and management. J Allergy Clin Immunol. 2017; 139(4):1111-1126. 2. Anvari S, Ruffner M A, Nowak-Wegrzyn A. Current and future perspectives on the consensus guideline for food protein-induced enterocolitis syndrome (FPIES). Allergol Int. 2024 April; 73(2):188-195. 3. Shah S, Grohman R, Nowak-Wegrzyn A. Food protein-induced enterocolitis syndrome (FPIES): Beyond the guidelines. J Food Allergy. 2023 Dec. 1; 5(2):55-64. 4. Sicherer S H, et al. Diagnosis and management of non-IgE-mediated food allergy in children. Pediatrics. 2014; 134(5):1064-1078. 5. Anvari S, Ruffner M A. Adult Food Protein-Induced Enterocolitis Syndrome. Front Allergy. 2022 May 26; 3:889879. 6. Akashi M, Kaburagi S, Kajita N, Morita H. Heterogeneity of food protein-induced enterocolitis syndrome (FPIES). Allergol Int. 2024 April; 73(2):196-205. 7. Cook V E, Connors L A, Vander Leek T K, Watson W. Non-immunoglobulin E-mediated food allergy. Allergy Asthma Clin Immunol. 2024 Dec. 19; 20(Suppl 3):70. 8. Mehr S, Frith K, Campbell D E. Food protein-induced enterocolitis syndrome: guidelines. Australasian Society of Clinical Immunology and Allergy (ASCIA). 2020. 9. Goswami R, Blázquez A B, Kosoy R, et al. Systemic innate immune activation in food protein-induced enterocolitis syndrome. J Allergy Clin Immunol. 2017; 139(6):1885-1896. 10. Chung H L, Hwang J B, Park J J, Kim S G. Expression of transforming growth factor-β1, transforming growth factor type I and II receptors, and TNF-α in the mucosa of the small intestine in infants with food protein-induced enterocolitis syndrome. J Allergy Clin Immunol. 2002; 109(1):150-154. 11. Pierucci-Alves F, Yi S, Schultz B D. Transforming growth factor-β1 induces tight-junction disruptions and loss of transepithelial resistance across porcine epithelial cells. Biol Reprod. 2012; 86(2):36. 12. Berin M C, Lozano-Ojalvo D, Agashe C, et al. Acute FPIES reactions are associated with an IL-17 inflammatory signature. J Allergy Clin Immunol. 2021; 148(3):895-901. 13. Mori F, Barni S, Cianferoni A, Pucci N, de Martino M, Novembre E. Cytokine expression in CD3+ cells in an infant with food protein-induced enterocolitis syndrome (FPIES): case report. Clin Dev Immunol. 2009:679381. 14. Gray H C, Foy T M, Becker B A, Knutsen A P. Rice-induced enterocolitis in an infant: TH1/TH2 cellular hypersensitivity and absent IgE reactivity. Ann Allergy Asthma Immunol. 2004 December; 93(6):601-5. 15. Adel-Patient K, Lezmi G, Castelli F A, Blanc S, Bernard H, Soulaines P, Dumond P, Ah-Leung S, Lageix F, de Boissieu D, Cortes-Perez N, Hazebrouck S, Fenaille F, Junot C, Dupont C. Deep analysis of immune response and metabolic signature in children with food protein induced enterocolitis to cow's milk. Clin Transl Allergy. 2018 Sep. 28; 8:38. 16. Muñoz-Bellido F J, Moreno E, Dávila I. Dupilumab: A Review of Present Indications and Off-Label Uses. J Investig Allergol Clin Immunol. 2022 Apr. 19; 32(2):97-115. 17. Ohshima Y, Tanaka Y, Tozawa H, Takahashi Y, Maliszewski C, Delespesse G. Expression and function of OX40 ligand on human dendritic cells. J Immunol. 1997 Oct. 15; 159(8):3838-48. 18. Lee S, Shan J, Aldossary H, Gaudreault V, Bazett M, Fixman E D. STAT6 inhibitory peptide reduces dendritic cell migration to the lymph nodes to control Th2 adaptive immunity in the mouse lung. Eur J Immunol. 2019 January; 49(1):157-169. 19. Bakker D S, van der Wal M M, Heeb L E M, Giovannone B, Asamoah M, Delemarre E M, Drylewicz J, Nierkens S, Boyman O, de Bruin-Weller M S, Thijs J L, van Wijk F. Early and Long-Term Effects of Dupilumab Treatment on Circulating T-Cell Functions in Patients with Moderate-to-Severe Atopic Dermatitis. J Invest Dermatol. 2021 August; 141(8):1943-1953. 20. Alpan O, Bachelder E, Isil E, Arnheiter H, Matzinger P. ‘Educated’ dendritic cells act as messengers from memory to naive T helper cells. Nat Immunol. 2004 June; 5(6):615-22. 21. Matzinger P. Tolerance, danger, and the extended family. Annu Rev Immunol. 1994; 12:991-1045. 22. Bellinghausen I, Weigmann B, Zevallos V, Maxeiner J, Reißig S, Waisman A, Schuppan D, Saloga J. Wheat amylase-trypsin inhibitors exacerbate intestinal and airway allergic immune responses in humanized mice. J Allergy Clin Immunol. 2019 January; 143(1):201-212. 23. Alpan O. Oral tolerance and gut-oriented immune response to dietary proteins. Curr Allergy Asthma Rep. 2001 November; 1(6):572-7. 24. Castro M, Corren J, Pavord I D, et al. Dupilumab suppresses type 2 inflammatory biomarkers across multiple atopic, allergic diseases. J Allergy Clin Immunol Pract. 2021; 9(3):1465-1473. 25. Pellefigues C, Mehta H, Prout M, et al. An OX-Tra'Ordinary Tale: The Role of OX40 and OX40L in Atopic Dermatitis. Cells. 2024; 13(7):587. 26. Wawrzyniak P, Akdis C A, O'Mahony L. Type 2 immunity in allergic diseases. Cell Mol Immunol. 2025. doi: 10.1038/s41423-025-01261-2. 27. Nowak-Węgrzyn A, Muraro A, Sopo S M, et al. Cytokine Expression in CD3+ Cells in an Infant with Food Protein-Induced Enterocolitis Syndrome (FPIES): Case Report. Int Arch Allergy Immunol. 2010; 152(1):70-74. 28. Caubet J C, Nowak-Wegrzyn A. Pathophysiology of Non-IgE-Mediated Food Allergy. Int Arch Allergy Immunol. 2012; 159(4):263-276. 29. Matsushita T, Ohtsuka Y, Ishida Y, et al. Successful use of dupilumab for egg-induced eosinophilic gastroenteritis with duodenal ulcer: a pediatric case report and review of literature. Allergol Int. 2024; 73(4):549-552. 30. Sampson H A. Rise of gastrointestinal-based food allergy. Ann Allergy Asthma Immunol. 2023; 131(6):747-749. 31. Wechsler M E, Guttman-Yassky E, Blauvelt A, et al. Phase 2b randomized trial of OX40 inhibitor telazorlimab for moderate-to-severe atopic dermatitis. J Allergy Clin Immunol. 2024; 153(5):1204-1215. 32. Nowak-Węgrzyn A, Sopo S M, Fiocchi A G, et al. FPIES: The dark side of food allergy. Allergol Immunopathol (Madr). 2017; 45(5):409-418. 33. Mehr S, Frith K, Campbell D E. Cytokine profile after oral food challenge in infants with food protein-induced enterocolitis syndrome. J Allergy Clin Immunol. 2016; 138(4):1199-1202. 34. Ng Q X, Soh A Y S, Loke W, Lim D Y, Yeo W S. The role of inflammation in irritable bowel syndrome (IBS). J Inflamm Res. 2018 Sep. 21; 11:345-349. 35. Chogle A, Mintjens S, Saps M. Pediatric IBS: an overview on pathophysiology, diagnosis and treatment. Pediatr Ann. 2014 April; 43(4): e76-82. 36. Nee J, Lembo A. Review Article: Current and future treatment approaches for IBS with diarrhoea (IBS-D) and IBS mixed pattern (IBS-M). Aliment Pharmacol Ther. 2021 December; 54 Suppl 1: S63-S74. 37. Levine J, Islam R K, Mo L, Kim B S, Talia J, Lipner S R, Ungar B. Dupilumab in Atopic Dermatitis Linked to Reduced Irritable Bowel Syndrome Risk: A Real-World Cohort Study. Allergy. 2025 Jun. 25. doi: 10.1111/all.16637. Epub ahead of print. PMID: 40560741.

1-56. (canceled)

57. A method of treating a food-triggered gastrointestinal disorder in a human subject in need thereof, wherein the subject has Food Protein-Induced Enterocolitis Syndrome (FPIES) or exhibits symptoms meeting the Rome IV criteria for irritable bowel syndrome (IBS), comprising:
administering to the subject a therapeutically effective amount of an interleukin-4 receptor alpha (IL-4Rα) antagonist.

58. The method of claim 57, wherein FPIES or IBS is associated with one or more of diseases selected from the group consisting of:
atopic dermatitis;
type 2 asthma;
eosinophilic esophagitis;
chronic spontaneous urticaria;
prurigo nodularis;
chronic rhino sinusitis with polyps (CRSwNP);
food triggered IBS; and
IBS.

59. The method of claim 57, wherein the IL-4Rα antagonist is a monoclonal antibody that binds and blocks IL4 and IL13 signaling through the IL-4Rα receptor.

60. The method of claim 57, wherein the IL-4Rα antagonist is
dupilumab, administered subcutaneously, wherein the dosage is optionally at about 200-600 mg every two or four weeks in an adult or weight-adjusted equivalent in a child; or about 200-300 mg;
rademikibart (CBP-201), administered subcutaneously, wherein the dosage is optionally at 150-300 mg every one to four weeks; or
manfidokimab (AK120), administered subcutaneously, wherein the dosage is optionally dosed at 200-300 mg every two weeks.

61. A method of treating a food-triggered gastrointestinal disorder in a human subject in need of treatment, wherein the disorder is selected from Food Protein-Induced Enterocolitis Syndrome (FPIES); or Rome IV criteria for irritable bowel syndrome (IBS), including
administering a therapeutically effective amount of an OX40 or OX40L pathway inhibitor.

62. The method of claim 61, wherein the OX40 or OX40L pathway inhibitor is a monoclonal antibody that binds OX40 or OX40L to prevent OX40 to OX40L interaction.

63. The method of claim 61, wherein the OX40 or OX40L pathway inhibitor is
rocatinlimab, administered subcutaneously, wherein the dosage is optionally at about 150 mg to 600 mg every 2 or 4 weeks (with or without an initial loading dose);
telazorlimab, administered subcutaneously, wherein the dosage is optionally at about 300 mg every two weeks or 300 mg every four weeks (following a loading dose), or about 600 mg every two weeks; or
amlitelimab, administered via subcutaneous injection, wherein the dosage is optionally at about 100 mg to 250 mg every four weeks (optionally with an initial loading dose of 200-500 mg).

64. A method of inducing oral tolerance to a food allergen in a subject with FPIES or IBS comprising:
(a) administering, to the subject a therapeutically effective amount of
an IL4Rα antagonist;
an OX40 or OX40L pathway inhibitor; or
both; and
(b) administering the food allergen in increasing amounts.

65. The method of claim 64,
wherein step (b) comprises gradual dietary introduction; and
wherein step (a) is administering the IL-4Rα antagonist given biweekly throughout the protocol; and
wherein tolerance is assessed by negative food challenge after at least 3-6 months of therapy.

66. The method of claim 57, wherein the administering step is only performed if the following steps (a) or (b) is positive and optionally if (c) is positive:
(a) testing the subject's blood sample to determine if ≥2% of events within either a CD11c⁺ myeloid dendritic-cell gate or a CD303 (BDCA-2)⁺ plasmacytoid dendritic-cell gate exhibiting OX40L signal above a positivity threshold set by a fluorescent-minus-one (FMO) control in the same assay of an age- and sex-matched healthy reference distribution measured on the same instrument, antibody clone, and gating strategy;
(b) testing the subject's blood sample to determine >3% of events within the T, B and >5% on NK cells exhibiting OX40 signal above a positivity threshold set by a fluorescent-minus-one (FMO) control in the same assay of an age- and sex-matched healthy reference distribution measured on the same instrument, antibody clone, and gating strategy; and
(c) optionally, contacting a portion of the blood sample with a candidate therapeutic agent which is optionally an OX40L blocking antibody; and observing whether the agent suppresses upregulation of OX40/OX40L.

67. The method of claim 66, wherein the benefit of the treatment is confirmed by determining that dendritic-cell OX40L or T, B or NK OX40 has decreased by ≥20% from a pretreatment value within 2-6 weeks after initiating therapy, the decrease being calculated using the same metric used to define OX40L or OX40 percent expression (percent-positive relative to FMO).

68. The method of claim 67, wherein the decrease is ≥30%, ≥40%, or ≥50%, measured as cell-surface OX40L % on dendritic cells or OX40% on T, B or NK cells by flow cytometry.

69. The method of claim 57, wherein administering step is determined by:
(i) maintaining the regimen if dendritic-cell OX40L decreases by ≥20% from baseline;
(ii) increasing dose frequency (shorten interval) if OX40L decreases by <20% and no clinical response is present;
(iii) discontinuing if OX40L increases by ≥10% and no clinical response is present; and
(iv) maintaining the regimen despite an OX40L decrease <20% if a clinical response is present.

70. A method of reducing a risk in a subject of a food-triggered gastrointestinal disorder including an acute FPIES or an acute IBS during an oral food challenge with a food or accidental ingestion of the food, comprising
(a) administering to the subject, a single dose of IL4Rα antagonist (such as dupilumab), OX40 or OX40L pathway inhibitor; and
(b) exposing the subject to the food.

71. A kit comprising,
(i) a vial of an IL-4Rα antagonist in a carrier; and
(ii) a vial of OX40 or OX40L pathway inhibitor in a pharmaceutically acceptable carrier.

72. A method for treating or diagnosing FPIES or IBS in a subject, comprising:
(a) obtaining a biological sample containing immune cells from the subject (such as peripheral blood);
(b) measuring immune cell (dendritic, T, B and NK cell) surface OX40 and OX40L by flow cytometry on the sample;
(c) exposing the sample ex vivo to one or more food antigens suspected of causing FPIES;
(d) measuring an expression of OX40 ligand (OX40L) by flow cytometry on dendritic cells in the sample, and/or the expression of OX40 on T, B or NK cells in the sample; and
(e) comparing said expression to a baseline or control sample not exposed to the one or more food antigens; and
(f) optionally treating the subject if an increase is detected in step (e).

73. A method of treating or identifying a human subject with FPIES or IBS who is likely to benefit from OX40/OX40L pathway inhibition after exposure to a suspect food antigen, comprising;
(a) performing an ex vivo T, B and NK cell activation assay on the subject's blood with the suspect food antigen, and
(b) detecting a level of OX40+ cells with a predetermined threshold (3% for T and B cells and 5% for NK cells) and OX40L with a predetermined threshold of 2% above baseline; and
(c) optionally treating the subject.
